(19) United States
(12) Patent Application Publication
(10) Pub. No.: US 2022/0176606 A1
(43) Pub. Date: Jun. 9, 2022

(54) APPARATUS, SYSTEM, AND METHOD FOR FORMING A COMPOUND FILM, AND APPARATUS HAVING A COMPOUND FILM

(71) Applicant: KCI LICENSING, INC., San Antonio, TX (US)

(72) Inventors: Christopher Brian LOCKE, Bournemouth (GB); Timothy Mark ROBINSON, Wimbourne (GB)

(21) Appl. No.: 17/435,891

(22) PCT Filed: Mar. 11, 2020
(86) PCT No.: PCT/US2020/021954

§ 371 Date: Sep. 2, 2021

Related U.S. Application Data
(60) Provisional application No. 62/816,351, filed on Mar. 11, 2019.

(51) Int. Cl. B29C 48/00; A61F 13/02; B29C 48/07; B29C 48/35; C09J 9/00

(52) U.S. Cl. CPC B29C 48/022; B29C 48/0011; A61F 13/0253; B29K 2075/00; B29C 48/35; B29C 48/0021; C09J 9/00; B29C 48/07

(57) ABSTRACT

This disclosure describes devices, systems, and methods related to a compound film. An exemplary compound film includes a first layer including a first polymer composition including polyurethane and a second layer including a second polymer composition removably coupled to the first layer. The compound film further includes light switchable adhesive coupled to the second layer and configured to transition from a first state to a second state, the light switchable adhesive has a first peel strength in the first state that is greater than a second peel strength of the light switchable adhesive in the second state. A third peel strength between the first layer and the second layer is less than the second peel strength between the light switchable adhesive in the first state and a bond site.

### CROSS-REFERENCE TO RELATED APPLICATIONS

This application claims priority to and the benefit of U.S. Provisional Application No. 62/816,351, filed Mar. 11, 2019, the contents of which is incorporated into the present application by reference in its entirety.

### TECHNICAL FIELD

Aspects of the present disclosure relate generally to a compound film, such as a compound film for use with a light switchable adhesive, and more specifically, but not by way of limitation, to an apparatus including the compound film and an apparatus, system, and method for forming the compound film.

### BACKGROUND

Light switchable (switched or light switched) adhesives are pressure sensitive adhesives that are “switchable” from a tacky state to a non-tacky or low-tack state in which the switched adhesive has a reduced peel strength relative to the peel strength of the adhesive before switching. To protect against accidental or inadvertent exposure, light switchable adhesive based hosts or devices commonly employ one or more light blocking layers and/or support or handling layers. To illustrate, a light blocking layer is commonly removably coupled to a non-light blocking layer to which the light switchable adhesive is applied. The non-light blocking layer may act as a host or support layer for the light switchable adhesive, and the light blocking layer is removed from the non-light blocking layer to activate the light switchable adhesive for easy removal.

In medical light switchable adhesive applications, high breathability of the layers is generally desired to reduce risk of maceration. Conventional light switchable adhesive applications typically use polyurethane (PU) films as a host layer (e.g., a non-light blocking film or layer) for a light switchable adhesive because of the high breathability of polyurethane (PU), as compared to other polymer materials such as polyethylene (PE). However, bonding films of similar types of polymer materials (e.g., PU/PU films) by a co-extrusion process produces a bond (e.g., peel strength) that is greater than a bond created by the light switchable adhesive. A co-extrusion process forms both films of polymer in-line, and then bonds both extruded films together. The similar chemical nature of the similar polymer materials of each film creates a relatively high bond strength, as compared to adhesives, when bonded together. Thus, when attempting to remove the light blocking layer from the non-light blocking layer, the light switchable adhesive bond would fail first and the compound film itself would detach from the patient before the light blocking layer is removed from the non-light blocking layer. Accordingly, co-extruded polyurethane films are not suitable hosts for light switchable adhesive.

Thus, some conventional light switchable adhesive applications use an adhesive or hot melt film to bond layers of similar types of materials. However, such adhesive or hot melt film acts as a barrier to air and moisture, and therefore reduces breathability and wearability. In other conventional light switchable adhesive applications, when polyurethane (PU) is used for a non-light blocking layer, the light blocking layer is another, dissimilar material, such as polyethylene (PE). However, polyethylene (PE) has a lower breathability as compared to polyurethane (PU), which can reduce wear time and lead to increased risk of maceration as compared to polyurethane (PU) films.

Therefore, each of the conventional light switchable adhesive applications are limited in use and/or effectiveness. As a result of the issues of each of the conventional light switchable adhesive applications, medical devices that incorporate such conventional compound films and light switchable adhesive can be painful to apply, use, and remove, have reduced wear times, and can cause skin maceration and damage.

### SUMMARY

This disclosure describes compound films and devices, systems, and methods related to forming and/or using compound films. In some implementations, a compound film may include two layers of similar polymer materials or the same polymer material. For example, a particular compound film may include a non-light blocking layer of polyurethane (PU) coupled to a light blocking layer of PU. Additionally, the system and methods described herein enable control and design of a bond strength (e.g., peel strength) between different layers of the compound films. Accordingly, such compound films can be used with, e.g., can include, light switchable adhesives (LSA), and such compound films can be used in the medical field to increase breathability and wearability of a bandage, closure, or dressing. To illustrate, a peel strength between similar polymer materials, e.g., two layers of polyurethane, can be reduced to be less than a peel strength of the LSA. Furthermore, medical devices including such compound films can be formed more easily and with reduced layers as compared to conventional compound films that use dissimilar material, such as medical LSA compound films having a polyethylene (PE) layer. Thus, manufacturing costs can be reduced and patient use and comfort can be increased by using less layers or films.

A light switchable adhesive (often referred to as a switched or light switched adhesive) is a pressure sensitive adhesive that is “switchable” from a tacky state (e.g., a first state) to a non-tacky or low-tack state (e.g., a second state) in which the light switchable adhesive has a reduced peel strength relative to the peel strength of the first state of the light switchable adhesive before switching. To illustrate, light, such as ultraviolet light, triggers (e.g., activates) cross linking in the light switchable adhesive which effectively decreases the bond (and peel strength) of the light switchable adhesive and enables a component of a wound closure device to decouple from a tissue site with reduced force. After the light switchable adhesive is switched from the first state to the second state by crosslinking, the light switchable adhesive becomes brittle and fragile, and the light switchable adhesive cannot be “uncrosslinked” or “unswitched.”

An exemplary compound film includes a first layer of a first polymer composition including polyurethane and a second layer of a second polymer composition removably coupled to the first layer. The compound film further includes light switchable adhesive coupled to the second layer and configured to transition from a first state to a second state, the light switchable adhesive has a first peel strength in the first state that is greater than a second peel strength of the light switchable adhesive in the second state. A third peel strength between the first layer and the second layer is less than the second peel strength between the light switchable adhesive in the first state and a bond site. In a particular implementation, the first polymer composition and the second polymer composition each comprise a same majority material. For example, each of the first polymer composition and the second polymer composition include polyurethane as a largest ingredient by weight or greater than 50 percent by weight.

In some implementations, the compound films described herein are manufactured by a film lamination process. For example, layers or films of polymer material of the compound film are received and are feed to rollers for compaction and bonding. The rollers apply pressure, and optionally heat, to the films of polymer material to bond together the films to form a compound film that is separable and has a low peel strength, as compared to co-extruded films. The peel strength of the compound film is sufficiently low that it can be used with light switchable adhesives.

In other implementations, the compound films described herein are manufactured by a extrusion-cast film process. For example, one of layers or films of polymer material of the compound film is formed or generated during the laminating process. To illustrate, a light blocking layer of the compound film is generated by a melt-blending process and an extruder and die thereof feed extrudate to be processed into a film of light blocking layer where rollers laminate the light blocking film and a non-light blocking film together by pressure, and optionally heat, to form compound film. Both of the film lamination process and the extrusion-cast film process produce bonds with a lower peel strength as compared to bonds formed by a co-extrusion process.

In some implementations, the first layer and the second layer are selectively bonded together in certain areas, are selectively bonded with a pattern, or both. For example, a patterned roller can be used to bond the layers in particular areas or zones and/or can be used to bond the films together with a particular pattern. To illustrate, a first portion (e.g., a peripheral portion) of the compound film may be bonded and a second portion (e.g., a central portion) of the compound film may not be bonded to reduce or control the peel strength, as compared to compound films without bonding zones and/or patterns. Additionally, or alternatively, an entirety or a portion of the compound film may be bonded with a patterned roller having a grid-like pattern of protrusions to control or reduce the peel strength as compared to compound films with non-patterned bonds. Accordingly, the peel strength of the compound film can be reduced or controlled, as compared to compound films with non-patterned bonds, such that the compound film can be used with conventional light switchable adhesives.

Additionally, or alternatively, the compound film may include perforations to increase breathability, facilitate removal of layers of the compound film, to control (e.g., reduce) a bond strength of the compound film, or a combination thereof. Reducing a bond strength of the compound film may include reducing an effectiveness of the light switchable adhesive by including perforations in the light switchable adhesive.

In some implementations, the compound film includes the light switchable adhesive in one or more patterns to control (e.g., reduce) a bond strength of the light switchable adhesive. For example, light switchable adhesive is applied to the non-light blocking layer in strips or a grid-like pattern. The light switchable adhesive may be applied selectively to portions of the non-light blocking layer, or may be applied to an entirety of the non-light blocking layer and portions of the light switchable adhesive may be removed.

Thus, the compound films of the present disclose are configured to have a lower peel strength between the first and second layers as compared to compound films of similar materials manufactured by co-extrusion. Accordingly, such compound films can be used with (or include) LSA. Additionally, the compound films enable multiple layers of the compound films, such as a light blocking layer and a non-light blocking layer (e.g., LSA host layer), to have increased breathability and wearability as compared to layers of conventional compound films that are used in LSA applications, i.e., compound films with sufficiently low peel strength to have a peel strength less than a peel strength of a bond created by the LSA between the LSA and a bond site (e.g., tissue site). As an illustrative example, the compound films described herein may enable a higher water vapor transfer rate and/or a higher oxygen transfer rate (e.g., permeability) as compared to compound films that include one high breathability layer and one relatively lower breathability layer, such as PE/PU films. Therefore, the compound films described herein are suitable for use in medical devices, such as bandages, drapes, dressings, and wound closures. The compound films enable medical devices to have reduced layers and increased breathability as compared to conventional compound films, thereby avoiding or limiting maceration and tissue damage at a tissue site and patient discomfort. Accordingly, the compound films may enable improved wound care and therapy and increased wear times of medical device, thereby advancing patient comfort and confidence in the treatment.

Some embodiments of the present apparatuses (e.g., a compound film) comprise: a first layer of a first polymer composition including polyurethane; a second layer of a second polymer composition, the second layer removably coupled to the first layer; and a light switchable adhesive coupled to the second layer and configured to transition from a first state to a second state, the light switchable adhesive has a first peel strength in the first state that is greater than a second peel strength of the light switchable adhesive in the second state, wherein a third peel strength between the first layer and the second layer is less than the second peel strength between the light switchable adhesive in the first state and a bond site.

In some of the foregoing embodiments of the present apparatuses, the light switchable adhesive includes or corresponds to a coating of light switchable adhesive in contact with the second layer, and the first polymer composition and the second polymer composition are polyurethane based. In some implementations, the first polymer composition and the second polymer composition are polyurethane blends.

In some of the foregoing embodiments of the present apparatuses, a peel strength between the first layer and the second layer is between 0.5 N/25 mm to 3 N/25 mm. In some implementations, the peel strength between the first layer and the second layer is between 1.5 N/25 mm to 2.5 N/25 mm. Additionally, or alternatively, a peel strength between the second layer and the light switchable adhesive is greater than 3 N/25 mm.

In some of the foregoing embodiments of the present apparatuses, the light switchable adhesive is configured to generate a peel strength of greater than 3 N/25 mm between the light switchable adhesive and a tissue site within 2 hours after application of the light switchable adhesive to the tissue site.

In some of the foregoing embodiments of the present apparatuses, the first layer is in direct contact with the second layer, and the light switchable adhesive is in direct contact with the second layer. Additionally, or alternatively, the first layer is opaque and the second layer is optically transparent.

In some of the foregoing embodiments of the present apparatuses, the first layer is configured to block or filter UV light to blue light, and the second layer is configured to pass UV light to blue light, or both. In some implementations, the second layer is configured to diffuse UV light to blue light. In other implementations, the second layer is configured to pass UV visible light, and the first layer is configured to block or filter visible light.

In some of the foregoing embodiments of the present apparatuses, the present apparatuses further comprise: a support layer of a third polymer material coupled to the first layer, the support layer having a first rigidity that is greater than a second rigidity of the first layer; and a cover film removably coupled to the light switchable adhesive. In some implementations, the first layer is included in a drape, a bandage, a wound closure device, a therapy system adhesive, or a combination thereof.

In some of the foregoing embodiments of the present apparatuses, the light switchable adhesive, the first layer, the second layer, or a combination thereof, define a plurality of perforations. In some implementations, the light switchable adhesive comprises a pattern of light switchable adhesive.

Some embodiments of the present methods of manufacturing a compound film comprise: providing a first film of a first polymer composition including polyurethane; providing a second film of a second polymer composition; and applying a light switchable adhesive to the second film of the compound film, the light switchable adhesive configured to transition from a first state having a first peel strength in the first state to a second state having a second peel strength, the first peel strength greater than the second peel strength, and a third peel strength between the first layer and the second layer is less than the second peel strength between the light switchable adhesive in the first state and a bond site.

In some of the foregoing embodiments of the present methods, providing a first film includes feeding, by a first roller, the first film, and providing the second film includes feeding, by a second roller, the second film. In some implementations, laminating the first film and the second film to generate the compound film includes applying heat, pressure, or both, by a third roller.

In some of the foregoing embodiments of the present methods, the methods further comprise applying heat to the third roller by a heater distinct from the third roller. Additionally, or alternatively, the methods further comprise applying heat, by a heater distinct from the third roller, to the first film, the second film, or both. In some of the foregoing embodiments of the present methods, the methods further comprise cooling the compound film.

In some of the foregoing embodiments of the present methods, applying the light switchable adhesive to the second film includes applying a coating of light switchable adhesive by a roller, a slot die, or a spray nozzle. Additionally, or alternatively, applying the light switchable adhesive to the second film includes applying the light switchable adhesive in a pattern. In some of the foregoing embodiments of the present methods, the first polymer composition is substantially similar to the second polymer composition.

In some of the foregoing embodiments of the present methods, the methods further comprise: coupling a cover film to the light switchable adhesive; and coupling a support layer of a third polymer composition to the first film. In some implementations, the third polymer composition is different from the first polymer composition, and the support layer is coupled to the first film prior to the first film being laminated to the second film.

In some of the foregoing embodiments of the present methods, the methods further comprise forming perforations in the light switchable adhesive, the first film, the second film, or a combination thereof. Additionally, or alternatively, the methods further comprise, prior to providing the first film, receiving a roll of the first film, a roll of the second film, or both. In some of the foregoing embodiments of the present methods, providing the first film includes extrusion casting the first film.

Some embodiments of the present systems (e.g., a manufacturing system) comprise: one or more first rollers associated with a first film of a first polymer composition including polyurethane; and one or more second rollers associated with a second film of a second polymer composition. The one or more first roller, the one or more second rollers, or a combination thereof are configured to laminate the first film and the second film to form a compound film. A peel strength between the first film and the second film of the compound film is less than 8 N/25 mm.

In some of the foregoing embodiments of the present systems, the systems further comprise an applicator configured to apply a light switchable adhesive to the second film. In some of the foregoing embodiments of the present systems, the one or more first rollers, the one or more second rollers, or both, include a roller selected from the group consisting of a stainless steel roller, a Teflon roller, and a silicone coated roller. Additionally, or alternatively, at least one of the one of more first rollers, the one or more second rollers, or both, comprise a patterned roller.

In some of the foregoing embodiments of the present systems, the systems further comprise a controller configured to control at least one of the one of more first rollers, the one or more second rollers, or a combination thereof. Additionally, or alternatively, the systems further comprise an extrusion cast film system including an extruder and a die and configured to generate the first film or the second film.

In some of the foregoing embodiments of the present systems, the systems further comprise a coating system configured to apply a light switchable adhesive to the second film of the compound film. In some implementations, the coating system comprises a roller, a platten, or a die configured to apply the light switchable adhesive. Additionally, or alternatively, the coating system is configured to the apply the light switchable adhesive to the second film of the compound film in a pattern.

In some of the foregoing embodiments of the present systems, the systems further comprise a steam heater or an electric heating device configured to heat at least one of the one of more first rollers, the one or more second rollers, or a combination thereof. Additionally, or alternatively, the systems further comprise a perforation device configured to generate perforations in the first film, the second film, or both. In a particular implementation, the perforations are microperforations, and the perforation device comprises a roller or a press.

In some of the foregoing embodiments of the present systems, the one or more first rollers includes a rotary press, a compression roller, a heating roller, a chilling roller, a driving roller, a feeding roller, or a combination thereof.

Some embodiments of the present systems (e.g., a therapy system) comprise: a medical device according to any of the foregoing embodiments. In some implementations, the systems further comprise a therapy device coupled to the medical device and configured to provide therapy to the medical device. Additionally, or alternatively, the systems further comprise a UV light source configured to emit UV light to the light switchable adhesive on the compound film to transition the light switchable adhesive from first state to a second state. In some of the foregoing embodiments of the present systems, the medical device is a wound dressing, a bandage, or a wound closure device. In some implementations, the compound film corresponds to a light blocking layer and a drape layer of the wound dressing.

As used herein, the term “switchable” will be used to refer to adhesives which can be changed from a high tack and/or peel strength state to a low tack and/or peel strength state (e.g., non-tacky state). Recognizing that the expression “low tack and/or peel strength” is a relative term, it will be defined here as meaning a condition of a minimum reduction in tackiness which the adhesive reaches after switching from the high tack and/or peel strength state. The reduction in tack or peel force may be as great as 99% or as little as 30%. Typically, the reduction in tack or peel force is between 70% and 90%.

As used herein, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically. Additionally, two items that are “coupled” may be unitary with each other. To illustrate, components may be coupled by virtue of physical proximity, being integral to a single structure, or being formed from the same piece of material. Coupling may also include mechanical, thermal, electrical, communicational (e.g., wired or wireless), or chemical coupling (such as a chemical bond) in some contexts.

The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. As used herein, the term “approximately” may be substituted with “within 10 percent of” what is specified. Additionally, the term “substantially” may be substituted with “within [a percentage] of” what is specified, where the percentage includes 0.1, 1, or 5 percent; or may be understood to mean with a design, manufacture, or measurement tolerance. The phrase “and/or” means and or. To illustrate, A, B, and/or C includes: A alone, B alone, C alone, a combination of A and B, a combination of A and C, a combination of B and C, or a combination of A, B, and C. In other words, “and/or” operates as an inclusive or.

The terms “comprise” (and any form of comprise, such as “comprises” and “comprising”), “have” (and any form of have, such as “has” and “having”), and “include” (and any form of include, such as “includes” and “including”). As a result, an apparatus that “comprises,” “has,” or “includes” one or more elements possesses those one or more elements, but is not limited to possessing only those one or more elements. Likewise, a method that “comprises,” “has,” or “includes” one or more steps possesses those one or more steps, but is not limited to possessing only those one or more steps.

Any aspect of any of the systems, methods, and article of manufacture can consist of or consist essentially of—rather than comprise/have/include—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb. Additionally, it will be understood that the term “wherein” may be used interchangeably with “where.”

Further, a device or system that is configured in a certain way is configured in at least that way, but it can also be configured in other ways than those specifically described. The feature or features of one embodiment may be applied to other embodiments, even though not described or illustrated, unless expressly prohibited by this disclosure or the nature of the embodiments.

Some details associated with the aspects of the present disclosure are described above, and others are described below. Other implementations, advantages, and features of the present disclosure will become apparent after review of the entire application, including the following sections: Brief Description of the Drawings, Detailed Description, and the Claims.

### BRIEF DESCRIPTION OF THE DRAWINGS

A further understanding of the nature and advantages of the present disclosure may be realized by reference to the following drawings. The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1A is a side view of an example of a system for manufacturing compound films;

FIG. 1B is a side view of an example of a compound film;

FIG. 1C is a side view of an example of a patterned roller;

FIG. 2 is a side view of another example of a system for manufacturing compound films;

FIGS. 3A-3D are each a side view of an example of a compound film attached to tissue;

FIGS. 4A-4F are each a cross-sectional view of an example of a compound film;

FIGS. 5A-5I are each a top view of an example pattern for a patterned roller;

FIG. 5J is a diagram of an example of bonding zones of a compound film;

FIG. 6A is a diagram of an example of a therapy system including a compound film;

FIG. 6B is a diagram of an example of the compound film of the therapy system of FIG. 6A;

FIG. 7 is a block diagram of a manufacturing system for making components including compound films coated in light switchable adhesive;

FIG. 8 is a flowchart illustrating an example of a method of manufacturing compound films; and

FIG. 9 is a flowchart illustrating an example of another method of manufacturing compound films.

### DETAILED DESCRIPTION

As used herein, the terms “tissue site” and “target tissue” as used herein can broadly refer to a wound (e.g., open or closed), a tissue disorder, and/or the like located on or within tissue, such as, for example, bone tissue, adipose tissue, muscle tissue, neural tissue, dermal tissue, vascular tissue, connective tissue, cartilage, tendons, ligaments, and/or the like. The terms “tissue site” and “target tissue” as used herein can also refer to a surrounding tissue area(s) and/or areas of tissue that are not necessarily wounded or exhibit a disorder, but include tissue that would benefit from tissue generation and/or tissue that may be harvested and transplanted to another tissue location. The terms “tissue site” and “target tissue” may also include incisions, such as a surgical incision. In some implementations, “target tissue” may correspond or refer to a wound, and “tissue site” may correspond or refer to a tissue area(s) surrounding and including the target tissue. Additionally, the term “wound” as used herein can refer to a chronic, subacute, acute, traumatic, and/or dehisced incision, laceration, puncture, avulsion, and/or the like, a partial-thickness and/or full thickness burn, an ulcer (e.g., diabetic, pressure, venous, and/or the like), flap, and/or graft. A wound may include chronic, acute, traumatic, subacute, and dehisced wounds, partial-thickness burns, ulcers (such as diabetic, pressure, or venous insufficiency ulcers), flaps, grafts, and fistulas, for example.

The term “positive-pressure” (or “hyperbaric”) as used herein generally refers to a pressure greater than a local ambient pressure, such as the ambient pressure in a local environment external to a sealed therapeutic environment (e.g., an internal volume). In most cases, this positive-pressure will be greater than the atmospheric pressure at which the patient is located. Alternatively, the positive-pressure may be greater than a hydrostatic pressure associated with tissue at the tissue site. Unless otherwise indicated, values of pressure stated herein are gauge pressures. References to increases in positive-pressure typically refer to an increase in absolute pressure, and decreases in positive-pressure typically refer to a decrease in absolute pressure. Additionally, the process of increasing pressure may be described illustratively herein as “applying”, “delivering,” “distributing,” “generating”, or “providing” positive-pressure, for example.

The term “reduced-pressure” (and “negative-pressure” or “hypobaric”) as used herein generally refers to a pressure less than a local ambient pressure, such as the ambient pressure in a local environment external to a sealed therapeutic environment (e.g., an internal volume). In most cases, this reduced-pressure will be less than the atmospheric pressure at which the patient is located. Alternatively, the reduced-pressure may be less than a hydrostatic pressure associated with tissue at the tissue site. Unless otherwise indicated, values of pressure stated herein are gauge pressures. References to increases in reduced-pressure typically refer to a decrease in absolute pressure, and decreases in reduced-pressure typically refer to an increase in absolute pressure. Additionally, the process of reducing pressure may be described illustratively herein as “applying”, “delivering,” “distributing,” “generating”, or “providing” reduced-pressure, for example.

The term “fluid” may refer to liquid, gas, air, or a combination thereof. The term “fluid seal,” or “seal,” means a seal adequate to maintain a pressure differential (e.g., positive-pressure or reduced-pressure) at a desired site given the particular pressure source or subsystem involved. Similarly, it may be convenient to describe certain features in terms of fluid “inlet” or “outlet” in such a frame of reference. However, the fluid path may also be reversed in some applications, such as by substituting a reduced-pressure source (negative or hypobaric pressure source) for a positive-pressure source, and this descriptive convention should not be construed as a limiting convention.

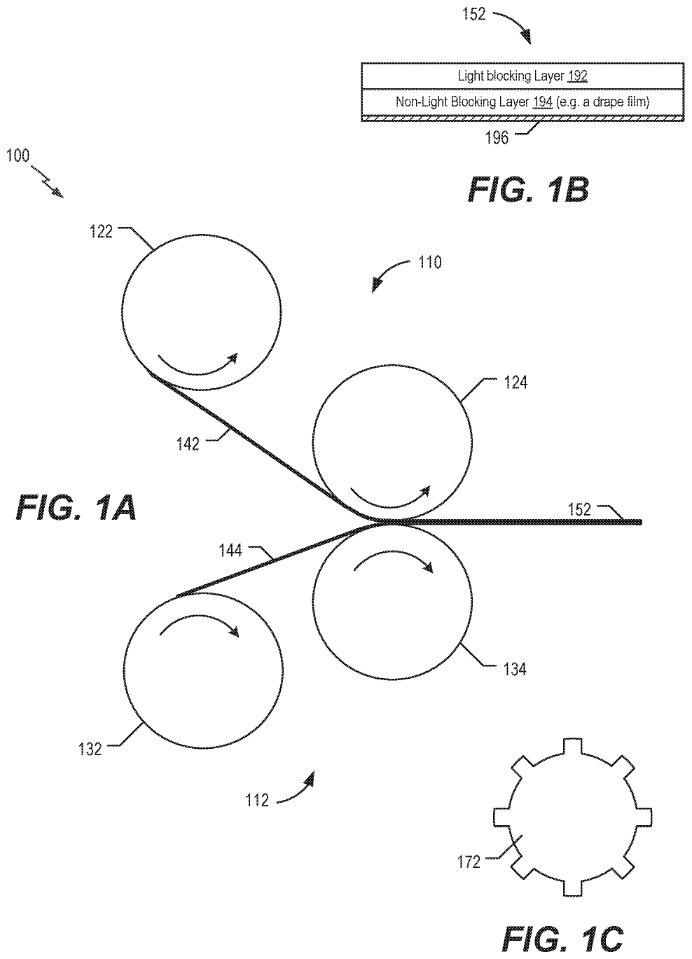

FIG. 1A shows a side view of an illustrative system 100 for manufacturing compound films. System 100 includes one or more first rollers 110 and one or more second rollers 112. System 100 is configured to form a compound film 152 including a first film 142 and a second film 144 where the first and second films comprise similar material. For example, system 100 may form a compound film 152 of two similar polymer materials that has a low bond strength between the two polymer materials, such as a low bond strength as compared to co-extruder compound polymer films. One such exemplary use of low bond strength compound films is as a host for light switchable adhesives.

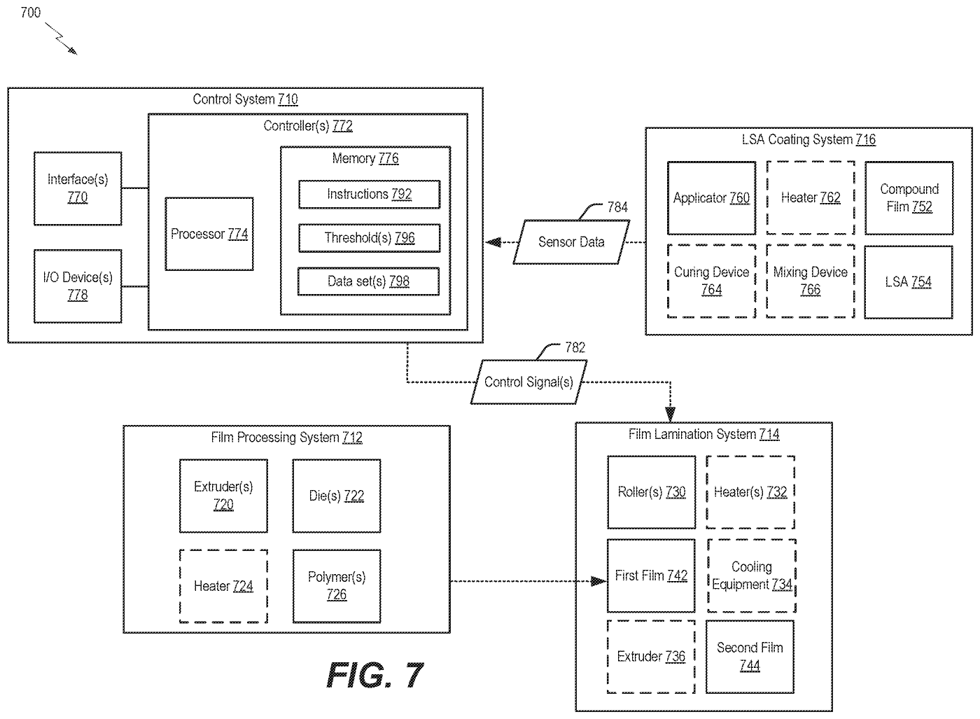

As illustrated in FIG. 1A, the one or more first rollers 110 include a first feed roller 122 and a first compression roller 124. First feed roller 122 may be configured to receive a roll of first film 142 and to feed or provide first film 142 to first compression roller 124. First compression roller 124 (e.g., a compression cylinder) is configured to receive the first film 142 from the first feed roller 122 and to apply pressure and/or heat to the first film 142 to bond (e.g., laminate) the first film 142 to the second film 144 to form the compound film 152. In some implementations, the compression roller 124 includes a heating device (e.g., a heat element) or is heated by a heating device, as described further with reference to FIG. 7. In other implementations, a distinct heating device or heating roller is used to heat the first film 142 to a temperature for bonding (e.g., lamination). Alternatively, the compression roller 124 generates heat from pressure and/or friction. The temperature of the compression roller 124 may be set based on a speed, a pressure, materials, and a desired bond strength.

Similarly, the one or more second rollers 112 include a second feed roller 132 and a second compression roller 134. Second feed roller 132 may be configured to receive a roll of second film 144 and is configured to feed or provide second film 144 to second compression roller 134. Second compression roller 134 (e.g., a compression cylinder) is configured to receive the second film 144 from the second feed roller 132 and is configured to apply pressure and/or heat to the second film 144 to bond (e.g., laminate) the second film 144 to the first film 142 to form the compound film 152. In some implementations, at least one of the one or more the first rollers 110, at least one of the one or more the second rollers 112, or a combination thereof may include or correspond to a stainless steel roller, a Teflon roller, or a silicone coated roller. In a particular implementation, a particular roller may be a combination roller. To illustrate, the particular roller may comprise a stainless steel roller with a silicone coating or may comprise a first potion that is stainless steel and a second portion that is Teflon.

Films 142, 144 are polymer films which have similar materials or the same material. For example, films 142, 144 may both be polyurethane (PU) films, polyethylene (PE) films, etc. In light switchable adhesive related applications, one of films 142, 144 is a light blocking film and the other of films 142, 144 is a non-light blocking film or light passing (e.g., transmitting) film, as described further with reference to FIG. 1B. In some implementations, second film 144 may include or correspond to a drape film. As described further herein, films 142, 142 may include an impermeable or semi-permeable, elastomeric material, as an illustrative, non-limiting example. In some implementations, compound film 152 may be liquid/gas (e.g., moisture/vapor) impermeable or semi-permeable.

Compound film 152 is configured to be separable and possesses a reduced bond strength (e.g., reduced peel strength) between films 142, 144 as compared to conventional co-extruded films of similar materials or of the same material. For example, a compound film 152 of two polyurethane (PU) based films has a lower bond strength between the films as compared to a coextruded compound film of two polyurethane (PU) based films. To illustrate, a compound film 152 of two polyurethane (PU) films or two polyurethane (PU) based films may have a bond strength of less than or equal to 7 Newtons (N) per 25 millimeter (mm) between the two PU films. In other implementations, a compound film 152 of two polyurethane (PU) films or two polyurethane (PU) based films may have a bond strength of less than or equal to any one of or between any two of: 0.5 N/25 mm, 1 N/25 mm, 1.5 N/25 mm, 2 N/25 mm, 2.5 N/25 mm, 3 N/25 mm, 4 N/25 mm, 5 N/25 mm, 6 N/25 mm, and 7 N/25 mm between the two PU films.

The compound film 152 may be post processed as described further herein. For example, the compound film 152 may be perforated and/or may be coupled, bonded to, or compound with one or more additional films or layers. In some implementations, a light switchable adhesive is applied to compound film 152, as described further with reference to FIG. 7.

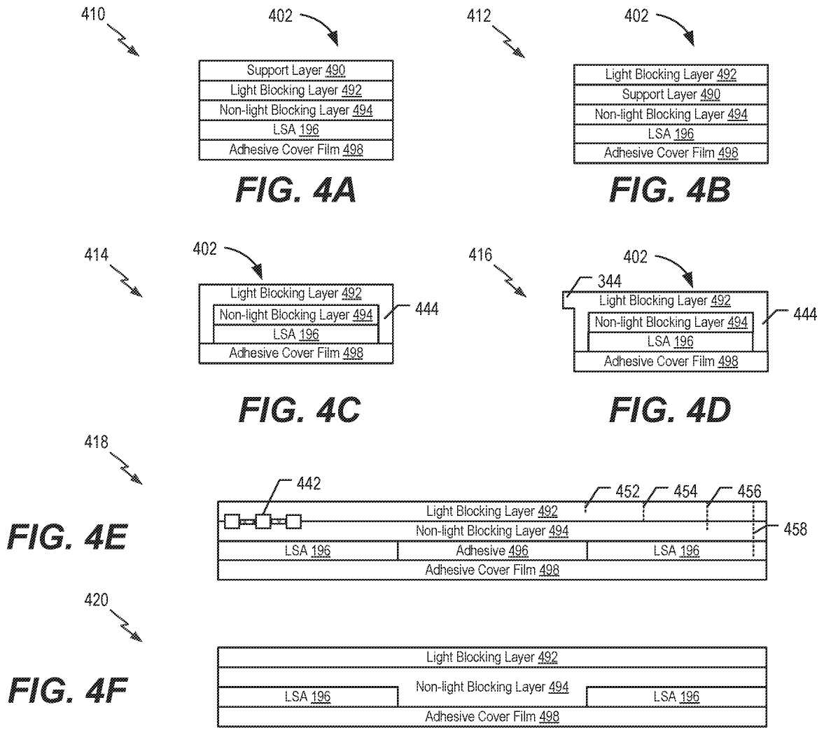

Referring to FIG. 1B, a side view of a particular example of compound film 152 including a light switchable adhesive is illustrated. In FIG. 1B, compound film 152 includes a removable protective film, referred to as a light blocking layer 192, a non-light blocking layer 194 (e.g., a light transmitting or passing layer), and a light switchable adhesive (LSA) 196. Light blocking layer 192 includes or corresponds to first film 142, and non-light blocking layer 194 includes or corresponds to second film 144. In other implementations, light blocking layer 192 includes or corresponds to second film 144, and non-light blocking layer 194 includes or corresponds to first film 142. In FIG. 1B, the light blocking layer 192 is in direct contact with the non-light blocking layer 194, and the LSA 196 is in direct contact with the non-light blocking layer 194. That is, compound film 152 does not include a support layer or an adhesive layer between the light blocking layer 192 and the non-light blocking layer 194. In other implementations, compound film 152 includes one or more additional layers, as described with reference to FIGS. 4A-4F.

Light blocking layer 192 is configured to be removed from non-light blocking layer 194 while non-light blocking layer 194 is bonded to a bond site, such as a tissue site (e.g., 620). Light blocking layer 192 is configured to block or filter light of a particular wavelength associated with activating the LSA 196, and non-light blocking layer 194 is configured to pass or transmit the light of the particular wavelength associated with activating the LSA 196. For example, the light blocking layer 192 may be configured to block or filter UV light to blue light wavelengths and/or the non-light blocking layer 194 may be configured to pass UV light to blue light wavelengths. To illustrate, the light blocking layer 192 is configured to block or filter light having a wavelength between 10 nanometers and 500 nanometers and/or the non-light blocking layer 194 is configured to pass light having a wavelength between 10 nanometers and 500 nanometers. In other implementations, the light which is blocked or filtered by the light blocking layer 192 and/or passed by non-light blocking layer 194 includes or corresponds to visible light, a portion of the visible light spectrum, UV light, a portion of the UV light spectrum, or a combination thereof. In a particular implementation, the light blocking layer 192 is opaque and the non-light blocking layer 194 is optically transparent.

In a particular implementation, light blocking layer 192 and non-light blocking layer 194 are configured to be permeable to air and water vapor, to enable tissue of tissue site to which the compound film 152 is bonded to “breathe.” Light blocking layer 192 and non-light blocking layer 194 have a bond that has a peel strength that is less than a peel strength of the LSA 196 in a first or high tack state. In a particular implementation, light blocking layer 192 includes a tab (e.g., 344) to enable easy removal of the light blocking layer 192 from the compound film 152. Tab may extend outwards and/or upwards from the compound film 152 to facilitate removal or light blocking layer 192 from non-light blocking layer 194.

Light blocking layer 192 and non-light blocking layer 194 of compound film 152 may include an impermeable or semi-permeable, elastomeric material, as an illustrative, non-limiting example. In some implementations, light blocking layer 192 and/or non-light blocking layer 194 are liquid/gas (e.g., moisture/vapor) impermeable or semi-permeable. Additionally, or alternatively, light blocking layer 192 and/or non-light blocking layer 194 include or are elastomeric material. “Elastomeric” means having the properties of an elastomer. For example, elastomer generally refers to a polymeric material that may have rubber-like properties. More specifically, an elastomer may typically have ultimate elongations greater than or equal to 100% and a significant amount of resilience. The resilience of a material refers to the material's ability to recover from an elastic deformation. Elastomers that are relatively less resilient may also be used as these elastomers. Examples of elastomers may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane (PU), EVA film, co-polyester, and silicones.

In some implementations, non-light blocking layer 194 is configured to diffuse light to LSA 196, such as light received from a top (e.g., when light blocking layer 192 is removed) and/or a side of non-light blocking layer 194. To illustrate, light received on a side of non-light blocking layer 194 is scattered as it passes through non-light blocking layer 194 to distribute the light to the LSA 196. Additionally, or alternatively, non-light blocking layer 194 may be formed of a thin, clear, flexible, breathable material with a high refractive index. One exemplary material for the cover film is polyurethane (PU).

LSA 196 is coupled to, such as in direct contact with, non-light blocking layer 194 of compound film 152 and is configured to generate a bond between the compound film 152 and a bond site, such as a tissue site of a patient. LSA 196 includes one or more photo initiators and is configured to switch states upon exposure to light of a particular spectrum or wavelength. The photo initiators are configured to absorb light (of particular spectrum or wavelength) and cross link with each other and/or free radicals to reduce tackiness, increase brittleness, increase fragility, reduce ductileness, change color, or a combination thereof. Thus, LSA 196 transitions from a first state (e.g., high tack state) to a second state (e.g., a low tack, no tack, or cross linked state) upon exposure to light. Transitioning from the first state to the second state may enable easy, pain and trauma free removal of the dressing and/or easy disconnection of a connection point. As an illustrative example, LSA 196 may be configured to create a bond between the LSA 196 and the bond site which has a peel strength of greater than or equal to 18 N in the first state and a peel strength of greater than or equal to 0.3 N in the second state. In some implementations, LSA 196 may include or correspond to a polyurethane (PU) or acrylic based light switchable adhesive. The peel strength values described herein are for similar conditions, such as temperature and humidity. Additionally, the peel strength values for the LSA or bond strength of the LSA, such as LSA 196, may be described in terms of a bond strength with human tissue at a particular time, such as two hours after application of the LSA.

In some implementations, LSA 196 includes UV photo initiators and is configured to absorb UV light (light from at least a portion of the UV spectrum) and switch states. In other implementations, LSA 196 includes visible light photo initiators and is configured to absorb visible light (light from at least a portion of the visible light spectrum) and switch states. LSA 196 may be applied to or disposed on non-light blocking layer 194 after compound film 152 is formed as described with reference to FIG. 7. In some implementations, LSA 196 is a coating or a pattern of coatings, as described further with reference to FIGS. 4E and 4F. Alternatively, LSA 196 may be formed with one or more films of the compound film 152, such as co-extruded with non-light blocking layer 194.

In some implementations, LSA 196 includes a UV marking additive. In a particular implementation, the UV marking additive includes or corresponds to an ultraviolet absorber (UV absorber). A UV absorber is a molecule used in organic or synthetic materials to absorb UV radiation. The UV absorbers are configured to absorb at least a portion of UV radiation of the UV spectrum and produce a visual indication, such as a color change. For example, UVA absorbers are configured to absorb UVA radiation, i.e., electromagnetic radiation having wavelengths between 300 and 400 nm. Additionally, or alternatively, one or more other layers of a compound film 152 may include a UV marking additive or another additive, such as a visible light additive. For example, light blocking layer 192 and/or non-light blocking layer 194 may include a marking additive. Such marking additives may produce a color change, produce text, produce a symbol, etc. to indicate light which may activate LSA 196 has been received.

In some implementations, LSA 196 has or is configured to provide a bond strength (e.g., peel strength) at least at or greater than, or substantially equal to any one of, or between two of: 3, 4, 5, 6, 8, 10, 12, 14, 16, 18, or 20 N, in the first state. The bond may be formed by LSA 196 between non-light blocking layer 194 and a bond site, such as target tissue of a tissue site. To illustrate, LSA 196 may have a bond strength as described above or may be applied such that compound film 152 has a bond strength as described above. In some implementations, the bond strength of the LSA 196 increases after application of LSA 196 to the bond site. For example, the bond strength of the LSA 196 may achieve (e.g., reach) a maximum bond strength between 30 minutes to 2 hours after application. Additionally, or alternatively, LSA 196 has or is configured to provide a bond strength (e.g., peel strength) at least at or greater than, or substantially equal to any one of, or between two of: 0.3, 0.5, 1, 2, 3, 4, 5, 6, 8, or 10 N, in the second state after being exposed to light.

Referring to FIG. 1A, although system 100 includes four rollers in FIG. 1A, in other implementations fewer than four rollers may be used. Alternatively, more than four rollers may be used, additional rollers and/or additional roller types may be used in place of one or more of the four rollers, or a combination thereof. For example, dedicated heating rollers, chilling rollers, may be used. Additionally, or alternatively, additional feed or compression rollers can be used. In some implementations, system 100 includes additional roller sets, such as one or more third rollers for an additional film and/or LSA 196.

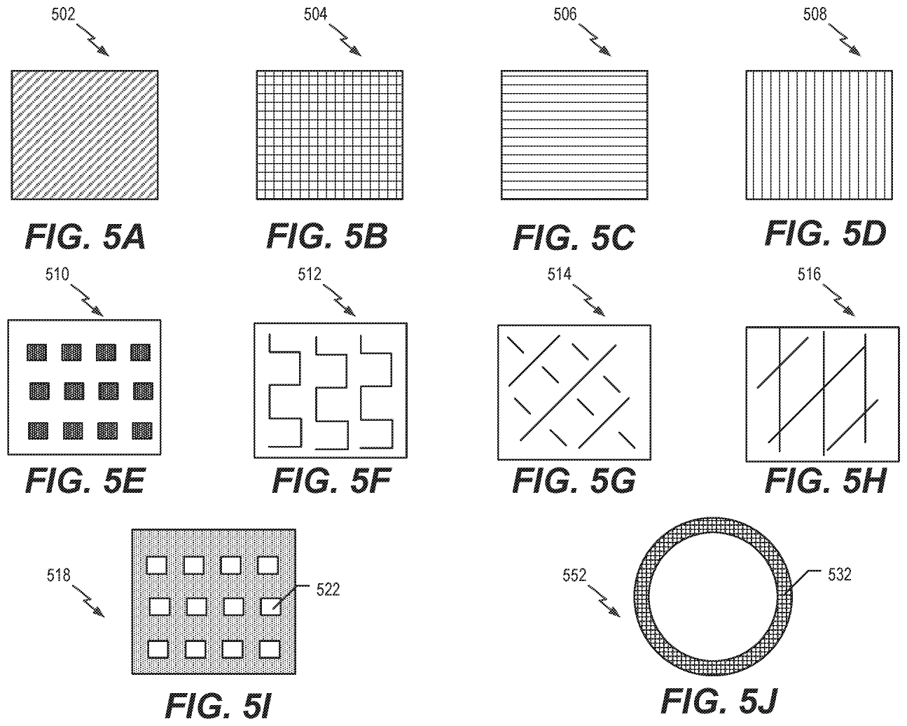

Referring to FIG. 1C, a patterned roller 172 is illustrated. Patterned roller 172 includes a pattern of raised protrusions on a rolling surface. Patterned roller 172 is configured to apply pressure and/or heat to only a portion of a film to bond portions (as opposed to a majority or an entirety) of the films 142, 144 to form compound film 152. To illustrate, raised surfaces (e.g., protrusions) of patterned roller 172 contact the film or films and apply pressure and/or heat to portions of the film and do not apply (or apply less pressure and/or heat) to other portions of the film. As an illustrative example, patterned roller 172 can be substituted for either of roller 124 or roller 134 to form selective bonding between films 142, 144, as further described with reference to FIGS. 5A-5H. Selective bonding provides another way to control (e.g., reduce) bond/peel strength between the first film 142 and the second film 144.

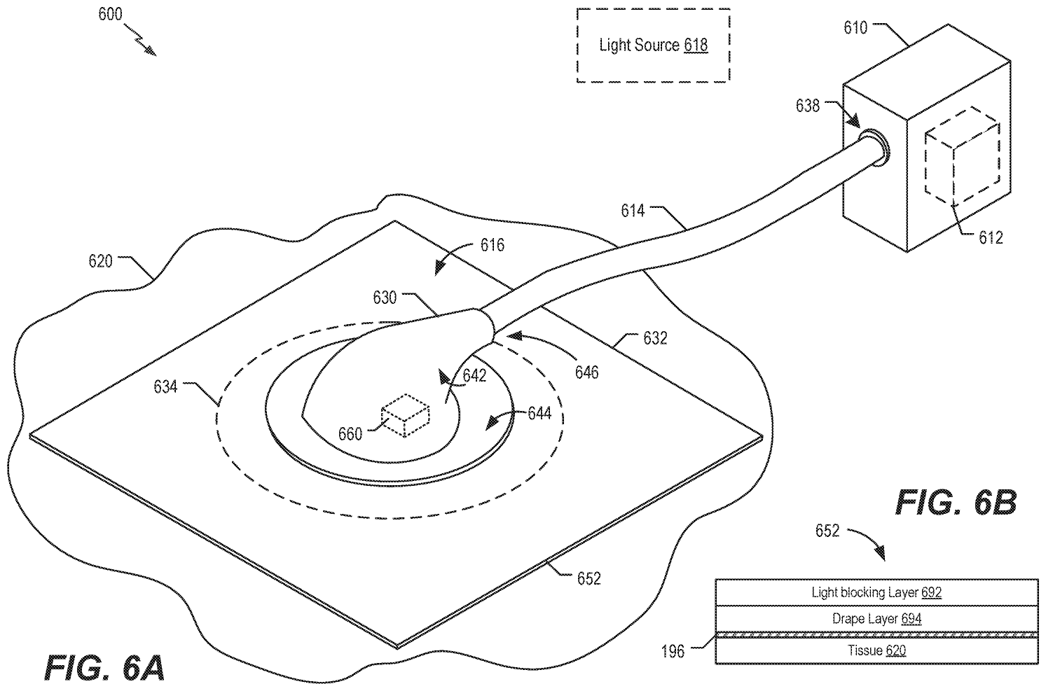

Compound film 152 may be configured to couple a bandage, a wound closure device, a dressing, and/or a drape, to provide a seal to create an enclosed space (e.g., an interior volume) corresponding to a tissue site. For example, compound film 152 may be configured to provide a fluid seal (i.e., provide a portion of fluid seal) between two components and/or two environments, such as between a sealed therapeutic environment and a local ambient environment. To illustrate, when coupled to a tissue site, compound film 152 is configured to maintain a pressure differential at the tissue site and/or keep fluids from permeating through the compound film 152, as described further with reference to FIG. 6A. Films 142, 144 may include an impermeable or semi-permeable, elastomeric material, as an illustrative, non-limiting example. In some implementations, compound film 152 may be liquid/gas (e.g., moisture/vapor) impermeable or semi-permeable.

During operation of system 100, a roll of film 142 is received at roller 122 and a roll of film 144 is received at roller 132. Roller 122 feeds film 142 to roller 124, and roller 132 feeds film 144 to roller 134. In some implementations, a portion of film 142 is manually fed from roller 122 to roller 124, a portion of film 144 is manually fed from roller 132 to roller 134, or both. In some implementations, roller 122 includes or is coupled to a motor configured to rotate the roller 122 to feed film 142 to roller 124. Additionally, or alternatively, roller 124 includes or is coupled to a motor configured to rotate the roller 124 to pull film 142 from roller 122.

As films 142, 144 are feed to or received at rollers 124, 134, rollers 124, 134 apply pressure to bond films 142 to 144 to form compound film 152. In some implementations, one or more of films 142, 144 are heated prior to being feed to or received at rollers 124, 134. For example, one or more of rollers 124, 134 include a heating device to heat the roller(s) 124, 134 or one or more of roller 124, 134 are associated with a heating device configured to apply heat to the film(s) 142, 144. As another example, one or more heating device are positioned between (e.g., interposed between) rollers 122, 132 and rollers 124, 134. Additionally, or alternatively, one or more of films 142, 144 are heated by one or more of rollers 124, 134. For example, one or more of rollers 124, 134 include a heating device to heat the roller(s) 124, 134. As another example, one or more of roller 124, 134 include or have a corresponding heating device configured to apply heat to the film(s) 142, 144.

In some implementations, system 100 includes patterned roller 172 in addition to rollers 124, 134 or in place of one or more of rollers 124, 134. Pattern roller(s) 172 apply pressure, and optionally heat, to a portion of films 142, 144 or compound film 152. Thus, portions of compound film 152 may have different bond strengths as compared to other portions of compound film 152. To illustrate, portions of compound film 152 pressed together by one or more pattern rollers 172 have a higher bond strength as compared to portions of compound film 152 not pressed together by one or more pattern rollers 172.

In some implementations, compound film 152 is cooled after bonding or forming, i.e. cooled after being pressed together by rollers 124, 134. For example, system 100 includes one or more cooled or chilled rollers configured to cool (i.e., remove heat from) compound film 152. To illustrate, one or more chilled rollers are received compound film 152 from rollers 124, 134 and reduce a temperature of compound film 152. As another example, compound film 152 is quenched in a water bath or cold fluid is applied to compound film 152.

Films 142, 144 of compound film 152 are removable/separable, i.e. are designed to be removed from each other during operation of the compound film 152. Removal of film 142 or 144 of compound film 152 from the other film is described further with reference to FIG. 6A.

In some implementations, a compound film includes a first layer of a first polymer composition including polyurethane and a second layer of a second polymer composition removeably coupled to the first layer. The compound film further includes a light switchable adhesive coupled to the second layer and configured to transition from a first state to a second state. The light switchable adhesive has a first peel strength in the first state that is greater than a second peel strength of the light switchable adhesive in the second state. A third peel strength between the first layer and the second layer is less than the second peel strength between the light switchable adhesive in the first state and a bond site.

In a particular implementation, the second polymer composition includes the same majority polymer, by weight percent, and/or includes a substantially similar polymer composition. To illustrate, each of the first polymer composition and the second polymer composition may include similar polymer materials and/or additives. Specifically, the second polymer composition includes two or more materials of the first polymer composition and includes a first concentration (e.g., by weight) of the two or more materials within plus or minus 20 percent of a second concentration of the two or more materials of the first polymer composition.

Thus, system 100 describes a film-to-film lamination system for manufacture (e.g., commercial manufacture of improved compound film. The compound films, such as, compound film 152, have a lower peel strength as compared to co-extruded compound films of similar materials such that compound film 152 can be used with or include LSA 196. Additionally, compound film 152 enables multiple layers of compound film 152, such as light blocking layer 192 and non-light blocking layer 194 (e.g., LSA 196 host layer) to have an increased breathability and wearability as compared to conventional co-extruded compound films and adhesively bonded compound films. For example, compound film 152 enables a higher water vapor transfer rate and/or a higher oxygen transfer rate (e.g., permeability) as compared to compound films that include one high breathability layer and one relatively lower breathability layer, such as PE/PU films, or an adhesive barrier between layers. Therefore, compound film 152 is suitable for use in medical devices, such as bandages, drapes, dressings, and wound closures. Compound film 152 enables medical devices to have reduced layers and increased breathability as compared to conventional compound film, thereby avoiding or limiting maceration and tissue damage at tissue site and patient discomfort. Accordingly, compound film 152 may enable improved wound care and therapy, thereby advancing patient comfort and confidence in the treatment.

Additionally, system 100 may enable fine tuning or adjusting of the bond strength of the compound film, as described further with reference to FIG. 7. For example, applying more pressure, heat, compaction time, or a combination thereof, generally increases bond strength and peel strength of the compound film. Compaction time may be increased by decreasing roller rotation speed (rpm) and/or film feed speed. To illustrate, higher heat, pressure, and time enable more polymer material of the films 142, 144 to bond together, thus creating a stronger bond. Accordingly, system 100 can generate compound films with a broader range of uses, as compared to coextruded and adhesively bonded films.

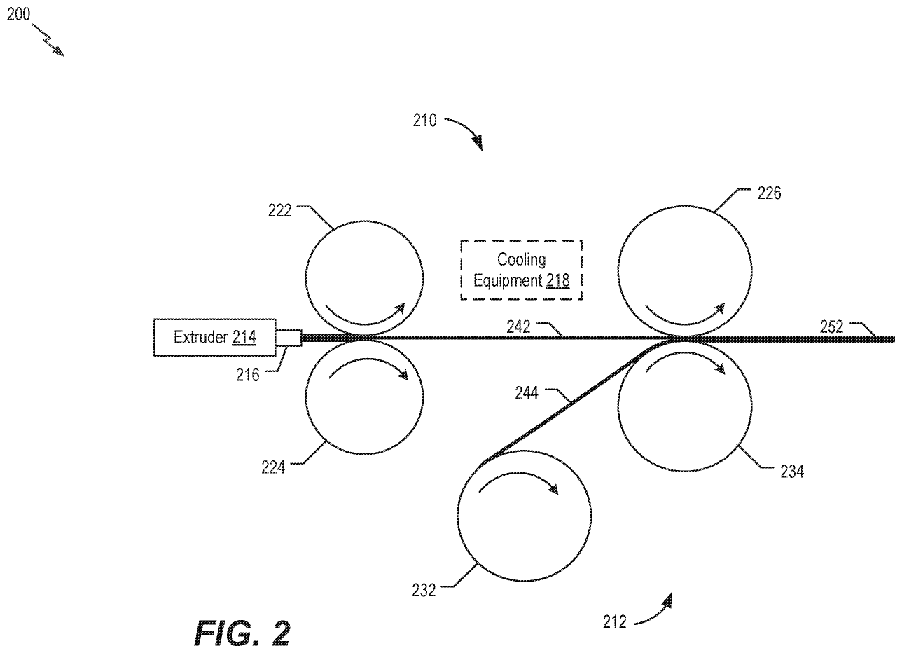

Referring to FIG. 2, a side view of another illustrative system 200 for manufacturing compound films is illustrated. System 200 includes one or more first rollers 210, one or more second rollers 212, an extruder 214, and a die 216. First rollers 210 may include or correspond to first rollers 110, and second rollers 212 may include or correspond to second rollers 112 of FIG. 1A System 200 is configured to form a compound film 252 including a first film 242 and a second film 244 where the first film 242 includes polyurethane and where one of the two films 242, 244 is extrusion cast. For example, system 200 may form a compound film 252 of two polyurethane (PU) films or two polyurethane (PU) based films with a low bond strength, i.e., a relatively lower bond strength as compared to a compound film formed by co-extrusion of two polyurethane (PU) films. One such exemplary use of low bond strength compound films is as a host for light switchable adhesives. As used herein, a polymer film, such as polyurethane film, includes the polymer (e.g., polyurethane), and a polymer based film, such as a polyurethane based film, includes at least 50.1 percent of the polymer by weight.

In some implementations, the first film 242 and the second film 244 may include substantially similar polymer compositions. To illustrate, each of the first polymer composition and the second polymer composition may include similar polymer materials and/or additives. In a particular implementation, the second polymer composition includes two or more materials of the first polymer composition and includes a first concentration (e.g., by weight) of the two or more materials within plus or minus 20 percent of a second concentration of the two or more materials of the first polymer composition.

Films 242, 244 may include or correspond to films 142, 144 of FIG. 1A. Compound film 252 may include or correspond to compound film 152 of FIG. 1A. As compared to system 100 of FIG. 1A, system 200 actively forms one of the two films 242, 244 during the lamination process, and as compared to co-extrusion systems, system 200 does not actively form both films 242, 244 during or as part of the lamination or combination process. Accordingly, compound film 252 has a lower bond strength as compared to co-extruded films of similar materials.

As illustrated in FIG. 2, the one or more first rollers 210 include multiple compression rollers 222-226. First and second compression rollers 222, 224 may be configured to receive an extruded film 240 (e.g., extrudate) from a die 216 of or incorporated with extruder 214 and compress the extruded film 240 to form the first film 242 and to feed or provide first film 242 to third compression roller 226. First and second compression rollers 222, 224 may further be configured to feed the first film 242 to third compression roller 226.

Third compression roller 226 (e.g., a compression cylinder) is configured to receive the first film 242 from the first and second compression rollers 222, 224 and is configured to apply pressure and/or heat to the first film 242 to bond (e.g., laminate) the first film 242 to the second film 244 to form the compound film 252. In some implementations, the third compression roller 226 includes a heating device or is heated by a heating device, as described further with reference to FIG. 7. In other implementations, a distinct heating device or heating roller is used to heat the first film 242 to a temperature for lamination. Additionally, or alternatively, the compression roller 224 may generate heat from pressure and/or friction. The temperature will depend on speed, pressure, material, and desired bond strength. Third compression roller 226 may include or correspond to compression roller 124.

The one or more second rollers 212 include a second feed roller 232 and a second compression roller 234, similar to FIG. 1A. Second rollers 232, 234 may include or correspond to second rollers 132, 134, respectively. Second feed roller 232 may be configured to receive a roll of second film 244 and to feed or provide second film 244 to second compression roller 234. Second compression roller 234 (e.g., a compression cylinder) is configured to receive the second film 244 from the second feed roller 232 and to apply pressure and/or heat to the second film 244 to bond (e.g., laminate) the second film 244 to the first film 242 to form the compound film 252.

Films 242, 244 are polymer films which have similar materials or the same material. For example, films 242, 244 may both be PU films, PE films, etc. In LSA related applications, one of films 242, 244 is a light blocking film and the other of films 242, 244 is a light transmitting film. For example, light which would otherwise activate the LSA is blocked or filtered by light blocking film and is transmitted by light transmitting film. In a particular implementation, the light transmitting film is configured to diffuse received light to LSA. Thus, when the light blocking film is removed from the light transmitting film, the light transmitting film can pass or diffuse light to the LSA.

Compound film 252 is configured to be separable and possesses a reduced bond strength (e.g., reduced peel strength) as compared to conventional co-extruded films of similar materials or of the same material. For example, a compound film 252 of two polyurethane (PU) based films has a lower bond strength as compared to a coextruded compound film of two polyurethane (PU) based films. To illustrate, a compound film 252 of two polyurethane (PU) films or two polyurethane (PU) based films may have a bond strength of less than or equal to 7 Newtons (N) per 25 millimeter (mm). In other implementations, a compound film 252 of two polyurethane (PU) films or two polyurethane (PU) based films may have a bond strength of less than or equal to any one of or between any two of: 0.5 N/25 mm, 1 N/25 mm, 1.5 N/25 mm, 2 N/25 mm, 2.5 N/25 mm, 3 N/25 mm, 4 N/25 mm, 5 N/25 mm, 6 N/25 mm, and 7 N/25 mm.

During operation of system 200, a roll of film 144 is received at roller 132. Roller 232 feeds film 244 to roller 234. In some implementations, a portion of film 242 is manually fed from rollers 222, 224 to roller 226, a portion of film 244 is manually fed from roller 232 to roller 234, or both. In some implementations, roller 222 and/or roller 224 includes a motor or is turned by a motor to feed film 242 to roller 226. Additionally, or alternatively, roller 226 includes a motor or is turned by a motor to pull film 242 from rollers 222, 224.

As films 242, 244 are feed to or received at rollers 226, 234, rollers 226, 234 apply pressure to bond films 242 to 244 to form compound film 252. In some implementations, one or more of films 242, 244 are heated prior to being feed to or received at rollers 226, 234. For example, one or more of rollers 226, 234 include a heating device to heat the roller(s) 226, 234 or one or more of roller 226, 234 are associated with a heating device configured to apply heat to the film(s) 242, 244. As another example, one or more heating device are positioned in between rollers 222, 224, 232 and rollers 226, 234. Additionally, or alternatively, one or more of films 242, 244 are heated by one or more of rollers 226, 234. For example, one or more of rollers 226, 234 include a heating device to heat the roller(s) 226, 234. As another example, one or more of roller 226, 234 include or have a corresponding heating device configured to apply heat to the film(s) 242, 244.

In some implementations, system 200 includes a patterned roller (e.g., 172) in addition to rollers 226, 234 or in place of one or more of rollers 226, 234. Patterned roller(s) 172 apply pressure, and optionally heat, to a portion of films 242, 244 or compound film 252. Thus, portions of compound film 252 may have different bond strengths as compared to other portions of compound film 252. To illustrate, portions of compound film 252 pressed together by one or more pattern rollers 172 have a higher bond strength as compared to portions of compound film 252 not pressed together by one or more pattern rollers 172.

In some implementations, first film 242 (e.g., extruded film) is cooled after bonding or forming, i.e. cooled after being extruded and pressed together by rollers 222, 224. For example, system 200 includes cooling equipment 218 (e.g., one or more cooled or chilled rollers) configured to cool (i.e., remove heat from/reduce a temperature of) first film 242. To illustrate, one or more chilled rollers receive the first film 242 from rollers 222, 224 and reduce a temperature of the first film 242. As another example, first film 242 is quenched in a water both or cold fluid is applied to first film 242.

Additionally, or alternatively, compound film 252 is cooled after bonding or forming, i.e. cooled after being pressed together by rollers 226, 234. For example, system 200 includes cooling equipment (e.g., second cooling equipment) to cool compound film 252. To illustrate, one or more chilled rollers are received compound film 252 from rollers 226, 234 and reduce a temperature of compound film 252. As another example, compound film 252 is quenched in a water both or cold fluid is applied to compound film 252.

Films 242, 244 of compound film 252 are removable/separable, i.e. are designed to be removed from each other during operation of the compound film 252. Removal of film 242 or 244 of compound film 252 from the other film is described further with reference to FIG. 6A. After compound film 252, is formed, LSA (e.g., 196) can be applied to the compound film as illustrated and described with reference to FIG. 7. LSA can be applied by a roller or a roller system, such as a compression roller 124 or a patterned roller 172, or by a die, platten, a rotary press, etc.

Thus, system 200 describes an extrusion-cast film system for manufacture (e.g., commercial manufacture of a compound film. As compared to system 100, system 200 actively forms a film or layer of compound film concurrently with forming/laminating the compound film. Accordingly, the in-line process of system 200 may include more components (e.g., extruder and die), but system 200 may have reduced costs as compared to system 100. In some implementations, less heating may be used in system 200 because the extruded film may already be above an ambient temperature.

Similar to compound film 152, compound film 252 is configured to have a lower peel strength between films 242, 244, as compared to co-extruded compound films of similar materials such that compound film 252 can be used with LSA (e.g., 196). Additionally, compound film 252 enables multiple layers of compound film 252, such as light blocking layer (e.g., 192) and non-light blocking layer (e.g., 194) to have and increased breathability and wearability as compared to conventional films. For example, compound film 252 enables a higher water vapor transfer rate and/or a higher oxygen transfer rate (e.g., permeability) as compared to compound films that include one high breathability layer and one relatively lower breathability layer, such as PE/PU films. Therefore, compound film 252 is suitable for use in medical devices, such as bandages, drapes, dressings, and wound closures. Compound film 252 enables medical devices to have reduced layers and increased breathability as compared to conventional compound film, thereby avoiding or limiting maceration and tissue damage at tissue site and patient discomfort. Accordingly, compound film 252 may enable improved wound care and therapy, thereby advancing patient comfort and confidence in the treatment.

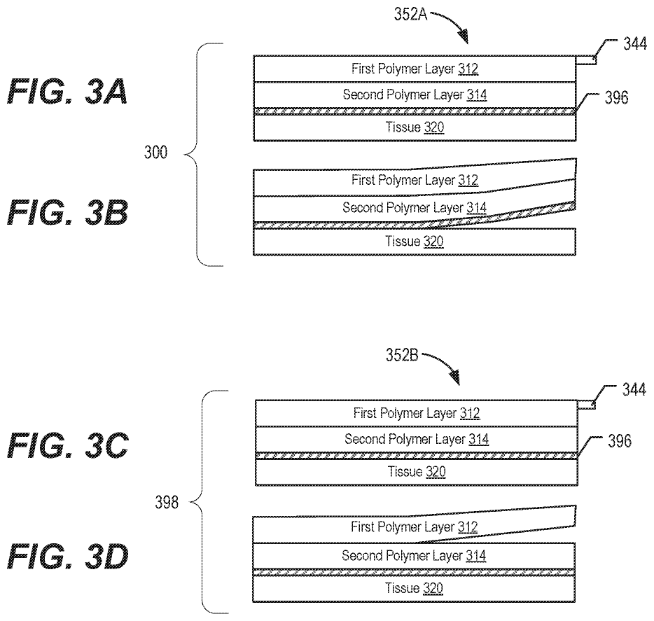

FIGS. 3A-3D illustrate examples of removing a compound film from a bond site, such as tissue 320. Referring to FIGS. 3A and 3B, an example 300 of removing a compound film 352A from tissue 320 is shown. Compound film 352A may include or correspond to compound film 152 or compound film 252. Tissue 320 may include or correspond to target tissue of a tissue site of a patient. FIG. 3A depicts a first state of compound film 352A attached to tissue 320 via LSA 396. FIG. 3B depicts a second state of compound film 352A during removal of compound film 352A.

Referring to FIG. 3A, compound film 352A includes first polymer layer 312, second polymer layer 314, and LSA 396. First polymer layer 312 may include or correspond to first film 142, 242, and second polymer layer 314 may include or correspond to second film 144, 244. In FIG. 3A, a bond strength between first polymer layer 312 and second polymer layer 314 is greater than a bond strength between LSA 396 and tissue 320 (and/or between LSA 396 and second polymer layer 314). Referring to FIG. 3B, a patient or care provider is attempting to remove first polymer layer 312 from second polymer layer 314. As shown in FIG. 3B, because the bond strength between first polymer layer 312 and second polymer layer 314 is greater than a bond strength between LSA 396 and tissue 320, the LSA 396 and the compound film 352A detach from tissue 320. Accordingly, compound film 352A is not an acceptable host for LSA 396.

Referring to FIGS. 3C and 3D, an example 398 of removing a compound film 352B from tissue 320 is shown. FIG. 3C depicts a first state of compound film 352B attached to tissue 320 via LSA 396. FIG. 3D depicts a second state of compound film 352B during removal of compound film 352B.

Referring to FIG. 3C, compound film 352B includes first polymer layer 312, second polymer layer 314, and LSA 396. First polymer layer 312 may include or correspond to first film 142, 242, and second polymer layer 314 may include or correspond to second film 144, 244. In FIG. 3A, a bond strength between first polymer layer 312 and second polymer layer 314 is greater than a bond strength between LSA 396 and tissue 320 (and/or between LSA 396 and second polymer layer 314). Referring to FIG. 3D, a patient or care provider is attempting to remove first polymer layer 312 from second polymer layer 314. In FIG. 3D, because the bond strength between first polymer layer 312 and second polymer layer 314 is less than a bond strength between LSA 396 and tissue 320 (and less than a bond strength between LSA 396 and second polymer layer 314), the first polymer layer 312 detaches from second polymer layer 314 prior to second polymer layer 314 detaching from LSA 396 and/or tissue 320.

In some implementations, a peel strength between the first polymer layer 312 and the second polymer layer 314 is between 0.5 N/25 mm to 3 N/25 mm. In a particular implementation, a peel strength between the first polymer layer 312 and the second polymer layer 314 is between 1.5 N/25 mm to 2.5 N/25 mm. Additionally, or alternatively, a peel strength between the second polymer layer 314 and the LSA 396 is greater than 3 N/25 mm. To illustrate, when LSA 396 is applied or disposed on the second polymer layer 314, the LSA 396 forms a bond with the second polymer layer 314 having a peel strength is greater than 3 N/25 mm in the first state. In a particular implementation, a peel strength between the second polymer layer 314 and the LSA 396 is greater than 8 N/25 mm.

In some implementations, the LSA 396 is configured to generate a peel strength of greater than 3 N/25 mm between the LSA 396 and a tissue 320 within 2 hours after application of the LSA 396 to the tissue 320. The tack level of the LSA 396 causes the LSA 396 to form a stronger bond with tissue 320 after application. Such a tack level allows for repositioning of the LSA 396 before the LSA 396 generates its maximum or operational bond strength. A cover film (e.g., 498) may protect LSA 396 from dust and/or debris and enable easier handling to ensure that LSA 396 forms its maximum or operating bond. In a particular implementation, the LSA 396 is configured to generate a peel strength of 3 N/25 mm to 8 N/25 mm or of greater than 8 N/25 mm between the LSA 396 and a tissue 320 within 2 hours after application of the LSA 396 to the tissue 320. Additionally, or alternatively, the LSA 396 is configured to form a bond between the LSA 396 and a tissue 320 having a peel strength of greater than 3 N/25 mm.

Accordingly, compound film 352B may be an acceptable host for LSA 396.

In some implementations, compound film 352A and/or 352B include a tab 344 to facilitate removal of first polymer layer from compound film (e.g., second polymer layer thereof). Formation of tab 344 is described with reference to FIGS. 4D and 7.

Referring to FIGS. 4A-4F, examples of cross-sections of a compound film 402 are shown. For example, compound film 402 may include or correspond to compound film 152 of FIGS. 1A and 1B, compound film 252 of FIG. 2, or compound film 352A, 352B of FIGS. 3A-3D. Referring to FIGS. 4A and 4B, exemplary positions of a support layer 490 are illustrated. Referring to FIG. 4A, a cross-section 410 of compound film 402 is shown. In FIG. 4A, compound film 402 includes support layer 490, light blocking layer 492, non-light blocking layer 494, LSA 196, and adhesive cover film 498. Layers 492, 494 may include or correspond to layers 192, 194 of FIG. 1B, respectively.

As illustrated in FIG. 4A, the support layer 490 is coupled, attached, or bonded to a first side (e.g., top side) of light blocking layer 492 of compound film 402. In FIG. 4B, a similar cross-section 412 is shown where the support layer 490 is coupled, attached, or bonded to a second side (e.g., bottom side) of light blocking layer 492 of compound film 402, such as a side associated with non-light blocking layer 494. Accordingly, as shown in FIG. 4B, support layer 490 is positioned between the light blocking layer 492 and the non-light blocking layer 494. Such a configuration enables easier removal of non-light blocking layer 494 from the light blocking layer 492 and may enable an entirety of light blocking layer 492 to be removed. For example, as compared to FIG. 4A, the configuration of the compound film 402 in FIG. 4B enables a user to remove light blocking layer 492 from compound film 402 without removing support layer 490. Thus, manufacturing can be simplified and costs reduced, by removal of a layer, and/or breathability of the tissue site can be increased by removal of layer that usually has a lower degree of breathability as compared to non-light blocking layer 494.

In such implementations, support layer 490 is configured to pass or transmit light such that light received by support layer 490 passes through compound film 402 to LSA 196. Additionally, as compared to conventional compound films with additional layers between a light blocking layer 492 and a non-light blocking layer 494, support layer 490 is made of similar polymer composition or material as compared to layers 492, 492. For example, each of layers 490-494 are polyurethane base layers or include polyurethane. Thus, support layer 490 can be bonded to one or more of layers 492, 494 by the system described in FIG. 1 or FIG. 2. Accordingly, compound film 402 of FIG. 4B still has increased breathability, similar to compound film 402 of FIG. 4A, as compared to conventional compound films that use one or more PE layers and/or adhesives between layers 492, 494.

Referring to FIGS. 4C and 4D, exemplary configurations of light blocking layers 492 are illustrated. Sidewalls 444 are illustrated in FIGS. 4C and 4D and a tab 344 is illustrated in FIG. 4D. Referring to FIG. 4C, a cross-section 414 of compound film 402 including a light blocking layer 492 that partially encompasses LSA 196 is shown. To illustrate, light blocking layer 492 has sidewalls (e.g., vertically arranged portions) that extend past the non-light blocking layer 494 and to the LSA 196. FIG. 4D illustrates a cross-section 416 of compound film 402 including a light blocking layer 492 that partially encompasses LSA 196 and includes a tab 344. Tab 344 may enable easier removal of light blocking layer 492, particularly sidewalls 444 thereof. The sidewalls 444 of FIGS. 4C and 4D block or filter light (e.g., ambient light) from reaching LSA 196 from the sides of compound film 402. Accordingly, such configurations may prevent or reduce unwanted activation and LSA 196, and thus LSA 196 may provide a stronger bond for a longer period of time.

In other implementations, sidewalls 444 extend to LSA 196 but not past or through LSA 196, i.e., LSA 196 is at least partially exposed on the sides of compound film 402. In such implementations where LSA 196 contacts light blocking layer 492, LSA 196 has a higher peel strength relative to non-light blocking layer 494 as compared to light blocking layer 492 because LSA 196 is applied to non-light blocking layer 494.

Referring to FIGS. 4E and 4F, exemplary configurations of patterns of LSA 196 on a compound film 402 are illustrated. Specifically, configurations where LSA 196 is coated or disposed on a portion of non-light blocking layer 494 (e.g., not coated or disposed on an entirety of non-light blocking layer 494). FIG. 4E illustrates a cross-section of 418 of a compound film 402 further including adhesive 496 (e.g., pressure sensitive adhesive) and including a pattern of LSA 196 and adhesive 496. In FIG. 4E, adhesive 496 is positioned in between sections or portions of LSA 196. Adhesive 496 may have a higher bond strength or a lower bond strength as compared to a bond strength of LSA 196. Thus, by selecting different adhesives (e.g., with different bond strengths) and/or using different amounts of adhesives, a bond strength of the compound film 402 can be tailored to meet design specifications.

Additionally, or alternatively, adhesive 496 (e.g., one or more portions thereof) may be replace with another non-adhesive material, such as a dummy material, or may be replace with nothing such that compound film includes a cavity defined by 196, 494, and 498. Accordingly, a bond strength of the compound film 402 can be reduced to meet design specifications.

FIG. 4E also illustrates an exemplary pattern 442 of selective bonding between light blocking layer 492 and non-light blocking layer 494. As illustrated in the example of FIG. 4E, two bonded sections are positioned in between three non-bonded sections (or relatively less bonded sections). The pattern of bonded sections may be generated by a patterned roller, such as patterned roller 172 of FIG. 1A. Examples of patterns of bonded sections are further illustrated in FIGS. 5A-5F. To illustrate, protrusions (and/or recesses) of a patterned roller apply different levels of pressure, and optionally heat, to different portions of the layers 492, 494 such that different levels of bonding occur between the two layers 492, 494. The different levels of bonding can reduce a peel strength between the two layers 492, 494.

FIG. 4E further illustrates exemplary perforations 452-458. One or more perforations 452-458 can be formed in one or more layers of compound film 402 to increase breathability, control a peel strength, or a combination thereof. For example, perforations 452 and 454 may increase breathability by improving breathability in light blocking layer 492. As another example, perforation 456 may increase breathability by improving breathability through layers 492 and 494. As yet another example perforation 458 may increase breathability in one or more layers of layers 196 or 492-496, such as from tissue site to ambient air. For example, perforations 452-458 may enable water vapor and/or oxygen to pass through the perforations 452-458. Including perforations may include or correspond to a surface or layer defining perforations or cavities.

In a particular example, the perforations 452-458 include or correspond to microperforations. The microperforations are sized such that light is not able to (or less than a threshold amount of light is able to) pass through the layers. For example, the microperforations have a diameter and/or a depth such that a majority of light entering the microperforations is captured in (e.g., absorbed in) or reflected by the microperforations.

Additionally, or alternatively, perforation 454 may reduce a peel strength between light blocking layer 492 and non-light blocking layer 494 and/or control removal of light blocking layer 492 from non-light blocking layer 494. Similar perforations (e.g., intralayer perforations) for layers 494 and 196 may reduce peel strength between layers 494 or 196 and adjacent layers and may control removal of light blocking layer 492 from non-light blocking layer 494 and of LSA 196 from tissue site. Such perforations may be formed in patterns and/or zones in the compound film 402, as described with reference to FIGS. 5A-5J, and perforations of different layers may be offset from each other to enable selective reduction in peel strength between two layers.

Referring to FIG. 4F, a cross-section 420 of a compound film 402 including a pattern of LSA 196 applied to recesses of non-light blocking layer 494 is illustrated. The recesses of non-light blocking layer 494 may be defined by different thicknesses in the non-light blocking layer 494 across cross-section 420. The recesses can be made during formation of non-light blocking layer 494 or formed after formation of non-light blocking layer 494, such as by machining or etching. The LSA 196 can be applied to recesses of non-light blocking layer 494 to reduce a bond strength between compound film 402 and tissue sites. Although LSA 196 is employed on edges of compound film 402, in other implementations, LSA 196 may be employed on an interior of compound film 402 to reduce exposure of the LSA 196 from the side. In other implementations, non-light blocking layer 404 does not include recesses and LSA 196 is applied to portions of non-light blocking layer 494 such that spaces or gaps are defined by discrete portions of sections of the LSA 196.

One or more features of FIGS. 4A-4F may be combined with one or more other features of FIGS. 4A-4F. For example, sidewalls 444 may be added to compound films 402 of FIGS. 4E and 4F. Although FIGS. 4A-4F illustrate adhesive cover film 498, adhesive cover film 498 is optional and may not be included in some implementations. Adhesive cover film 498 (e.g., an adhesive cover layer) is positioned over or coupled to LSA 196 to protect LSA 196 from activation, i.e., receiving light and transitioning to the second state, and from dust or contamination. Adhesive cover film 498 is configured to be removed prior to application of compound film 402 to tissue site and as such has a lower peel strength or bond strength to the LSA 196 than a peel strength or bond strength between the LSA 196 and the non-light blocking layer 194 when the LSA 196 is in the first state. Adhesive cover film 498 may be formed of a thin, clear, flexible, breathable material with a high refractive index. One exemplary material for adhesive cover film 498 is polyurethane (PU).

FIGS. 5A-5J illustrate various examples of patterns of patterned rollers, such a patterned roller 172 of FIG. 1A, for forming compound films, such as compound film 152, compound film 252, etc.

FIGS. 5A-5H illustrate various examples of patterns of patterned rollers including protrusions, i.e., a pattern of raised surfaces. In other implementations, one or more of the patterns of FIGS. 5A-5H may be formed in relief, i.e., inscribed into a surface of the patterned roller and corresponding to recesses of the patterned roller. FIG. 5I illustrates an example of a pattern of recesses of a patterned roller. FIG. 5J illustrates an example of a zone of selective bonding formed in a compound film by a roller or a patterned roller.

Referring to FIG. 5A, a first pattern 502 of a patterned roller is shown. First pattern 502 includes or corresponds to a diagonal pattern of protrusions. Referring to FIG. 5B, a second pattern 504 of a patterned roller is shown. Second pattern 504 includes or corresponds to a grid-like pattern of protrusions. To illustrate, the second pattern 504 includes a first arrangement of objects (e.g., lines) in a first direction (e.g., vertical) and a second arrangement of objects (e.g., lines) in a second direction (e.g., horizontal).

Referring to FIG. 5C, a third pattern 506 of a patterned roller is shown. Third pattern 506 includes or corresponds to a horizontal pattern of protrusions. Referring to FIG. 5D, a fourth pattern 508 of a patterned roller is shown. Fourth pattern 508 includes or corresponds to a vertical pattern of protrusions.

Referring to FIG. 5E, a fifth pattern 510 of a patterned roller is shown. Fifth pattern 510 includes or corresponds to another grid-like pattern of protrusions. To illustrate, the fifth pattern 510 includes a first arrangement of objects (e.g., square shapes) in a first direction (e.g., vertical) and a second arrangement of objects (e.g., square shapes) in a second direction (e.g., horizontal). In other implementations, different shapes may be used, such as circles, triangles, diamonds, rectangles, etc.

Referring to FIG. 5F, a sixth pattern 512 of a patterned roller is shown. Sixth pattern 512 includes or corresponds to a compound pattern of protrusions. To illustrate, the sixth pattern 512 includes a repeating pattern of objects (e.g., lines) that extend back in forth in multiple directions. As illustrated in FIG. 5F, the repeating pattern of lines extend in a first direction (e.g., vertical) and in a second direction (e.g., horizontal). In other implementations, different directions may be used, such as diagonal.

Referring to FIG. 5G, a seventh pattern 514 of a patterned roller is shown. Seventh pattern 514 includes or corresponds to a compound diagonal pattern of protrusions. Referring to FIG. 5H, an eighth pattern 516 of a patterned roller is shown. Eighth pattern 516 includes or corresponds to a compound pattern of protrusions.

Referring to FIG. 5I, a ninth pattern 518 of recesses 522 (e.g., a reverse or relief pattern) of a patterned roller is shown. Ninth pattern 518 includes or corresponds to a grid-like pattern of square shaped recesses. In other implementations, different shapes may be used, such as lines, circles, triangles, diamonds, rectangles, etc.

Referring to FIG. 5J, a zone 532 (e.g., area) of a compound film 552 is formed with a roller or a patterned roller is illustrated. Zone 532 corresponds to a particular portion of the compound film 552 where a roller or patterned roller bonded or selectively bonded one or more layers of the compound film 552 together. As illustrated in the example of FIG. 5J, the compound film 552 has a circular shape and the zone 532 of the compound film 552 formed with a patterned roller correspond to an annular rim portion (e.g., circumference portion or outer edge/rim) of the circular compound film 552. Thus, in the example illustrated in FIG. 5J, the zone 532 (outer rim) has a lower peel strength than the rest (e.g., interior) of the compound film 552. Accordingly, a patient or care provider can peel the outer edges of the compound film 552 more easily. When the zone 532 of the compound film 552 is formed with a roller and the rest of the compound film 552 is not formed with a roller or is formed with a patterned roller, the zone 532 (outer rim) has a higher peel strength than the rest (e.g., interior) of the compound film 552. Accordingly, a patient or care provider can detach the interior or central portion of compound film 552, such as by using perforations to outline zone 532, more easily and expose a portion of the LSA to light to enable safe and pain free removal of the compound film 552.

In some implementations, the compound film 552 may include multiple zones. In a particular implementation, each zone may have a corresponding pattern (e.g., may be formed by a different corresponding roller or patterned roller). In other implementations, an interior portion of the compound film 552 may be formed with a patterned roller to reduce a peel strength of the interior portion as compared to an exterior portion of the compound film 552. Thus, an edge or exterior portion of the compound film 552 may be more resistant to accidental separation, and may be more easily allow separation of the interior of the compound film 552.

Although FIGS. 5A-5J have been described with reference to patterns of patterned rollers, the patterns of protrusions and recesses of FIGS. 5A-5J can also correspond to patterns of light switchable adhesive. For example, light switchable adhesive can be selectively applied to form the patterns 502-518 illustrated in FIGS. 5A-5J. Alternatively, light switchable adhesive may be applied and then selectively removed to form the patterns 502-518. In addition, the patterns 502-518 illustrated in FIGS. 5A-5J can also correspond to patterns of perforations, such as the perforations 452-458 of FIG. 4E. Similarly, although FIG. 5I has been described with reference to a zone of selective bonding, the zone of selective bonding may include or correspond to a zone or light switchable adhesive or a zone of perforations. The zone of light switchable adhesives or perforations may include one of the patterns 502-518 of FIGS. 5A-5J.

Although FIGS. 5A-5I are illustrated as separate patterns or configurations, aspects of each pattern or configuration can be used separately or in combination with aspects of other patterns or configurations. For example, multiple patterns can be combined. Additionally, patterns, zones, and perforations may be used together. For example, a first particular pattern can be used for adhesives and a second particular pattern can be used for perforations. Furthermore, the patterns illustrated in FIGS. 5A-5I can modified based on the other patterns illustrated in FIGS. 5A-5I. For example, a particular pattern illustrated in FIGS. 5A-5I may be rotated in other implementations.

FIG. 6A shows a perspective view of an illustrative system 600 (e.g., a therapy system) for providing wound therapy. System 600 may include a compound film as described herein (e.g., 152, 252, 352A, 352B, 402, 552), a therapy device 610, a canister 612, a tube 614, a dressing 616, and a light source 618 (e.g., UV device). As an illustrative example, system 600 includes compound film 652 as part of dressing 616 (e.g., drape 632 thereof). System 600 is configured to provide therapy (e.g., oxygen therapy, positive-pressure therapy, negative-pressure therapy, or a combination thereof) at a tissue site 620 associated with a target area of a patient. For example, dressing 616 may be in fluid communication with tissue site 620 and may be in fluid communication with therapy device 610 via tube 614. In some implementations, system 600 may include one or more components commercially available through and/or from KCI USA, Inc. of San Antonio, Tex., U.S.A., and/or its subsidiary and related companies (collectively, “KCI”).

Therapy device 610 (e.g., a treatment apparatus) is configured to provide therapy to tissue site 620 via tube 614 and dressing 616. For example, therapy device 610 may include a pressure source (e.g., a negative-pressure source, such as a pump, or a positive-pressure source, such as a pressurized oxygen container, an oxygen concentrator, or an oxygen collector) configured to be actuatable (and/or actuated) to apply pressure differential relative to ambient conditions to dressing 616. As illustrative, non-limiting examples, positive-pressure applied to a tissue site may typically ranges between 5 millimeters mercury (mm Hg) (667 pascals (Pa)) and 30 mm Hg (4.00 kilo (k) Pa). Common therapeutic ranges are between 10 mm Hg (1.33 kPa) and 25 mm Hg (3.33 kPa). As illustrative, non-limiting examples, reduced-pressure applied to a tissue site may typically ranges between −5 millimeters mercury (mm Hg) (−667 pascals (Pa)) and −500 mm Hg (−66.7 kilo (k) Pa). Common therapeutic ranges are between −75 mm Hg (−9.9 kPa) and −300 mm Hg (−39.9 kPa).

In some implementations, therapy device 610 may alternate between providing positive-pressure therapy and negative-pressure therapy to the dressing 616, may provide positive-pressure therapy to a first portion of the dressing 616 and negative-pressure therapy to a second portion of the dressing 616, may provide no positive or negative pressure, or a combination thereof. In some such implementations, the therapy device 610 can provide positive-pressure therapy and negative-pressure therapy to the dressing 616 at the same time (e.g., partially concurrently).

As illustrated in FIG. 6A, therapy device 610 includes canister 612 to receive fluid from tissue site 620 or to provide fluid to tissue site 620. Although canister 612 is illustrated as being internal to and/or integrated with therapy device 610, in other implementations, canister 612 is external to therapy device 610, as illustrated and described with reference to FIG. 1A.

Therapy device 610 may also include one or more other components, such as a sensor, a processing unit (e.g., a processor), an alarm indicator, a memory, a database, software, a display device, a user interface, a regulator, and/or another component, that further facilitate positive-pressure therapy. Additionally, or alternatively, therapy device 610 may be configured to receive fluid, exudate, and or the like via dressing 616 and tube 614. Therapy device 610 may include one or connectors, such as a representative connector 638. Connector 630 is configured to be coupled to tube 614. Additionally, or alternatively, therapy device 610 may include one or more sensors, such a pressure sensor (e.g., a pressure transducer). The one or more sensors may be configured to enable therapy device 610 to monitor and/or sense a pressure associated with tube 614 and/or dressing 616.

Tube 614 includes one or more lumens (e.g., one or more through conduits), such as a single lumen conduit or multiple single-lumen conduits. Tube 614 (e.g., a least one of the one or more lumens) is configured to enable fluid communication between therapy device 610 and dressing 616. For example, fluid(s) and/or exudate can be communicated between therapy device 610 and dressing 616, and/or one or more pressure differentials (e.g., positive-pressure, negative pressure, or both) can be applied by therapy device 610 to dressing 616. As an illustrative, non-limiting illustration, tube 614 is configured to deliver at least pressurized oxygen from therapy device 610 to dressing 616 to establish positive-pressure. Communication of fluid(s) and application of a pressure differential can occur separately and/or concurrently.

In some implementations, tube 614 may include multiple lumens, such as a primary lumen (e.g., a positive-pressure/fluid lumen) for application of positive-pressure and/or communication of fluid, and one or more secondary lumens proximate to or around the primary lumen. The one or more secondary lumens (e.g., one or more ancillary/peripheral lumens) may be coupled to one or more sensors (of therapy device 610), coupled to one or more valves, as an illustrative, non-limiting example. Although tube 614 is described as a single tube, in other implementations, system 600 may include multiple tubes, such as multiple distinct tubes coupled to therapy device 610, dressing 616, or both.

As used herein, a “tube” broadly refers to a tube, pipe, hose, conduit, or other structure with one or more lumens adapted to convey fluid, exudate, and/or the like, between two ends. In some implementations, a tube may be an elongated, cylindrical structure with some flexibility; however, a tube is not limited to such a structure. Accordingly, tube may be understood to include a multiple geometries and rigidity. Tube 614 includes one or more lumens (e.g., one or more through conduits), such as a single lumen conduit or multiple single-lumen conduits. Tube 614 (e.g., a least one of the one or more lumens) is configured to enable fluid communication between therapy device 610 and dressing 616. For example, fluid(s) and/or exudate can be communicated between therapy device 610 and dressing 616, and/or one or more pressure differentials (e.g., positive-pressure, negative pressure, or both) can be applied by therapy device 610 to dressing 616. As an illustrative, non-limiting illustration, tube 614 is configured to deliver at least pressurized oxygen from therapy device 610 to dressing 616 to establish positive-pressure. Communication of fluid(s) and application of a pressure differential can occur separately and/or concurrently.

Dressing 616 includes a connector 630 (also referred to as a dressing connection pad or a pad), a drape 632, and a manifold 634 (also referred to as a distribution manifold or an insert). Drape 632 may be coupled to connector 630. To illustrate, drape 632 may be coupled to connector 630 via an adhesive, a separate adhesive drape over at least a portion of connector 630 and at least a portion of drape 632, or a combination thereof, as illustrative, non-limiting examples.

Drape 632 may be configured to couple dressing 616 at tissue site 620 and/or to provide a seal to create an enclosed space (e.g., an interior volume) corresponding to tissue site 620. For example, drape 632 may be configured to provide a fluid seal between two components and/or two environments, such as between a sealed therapeutic environment and a local ambient environment. To illustrate, when coupled to tissue site 620, drape 632 is configured to maintain a pressure differential (provided by a positive-pressure source or a negative-pressure source) at tissue site 620. Drape 632 may include a drape aperture that extends through drape 632 to enable fluid communication between device and target tissue. Drape 632 may be configured to be coupled to tissue site 620 via an adhesive, such as a medically acceptable, pressure-sensitive adhesive that extends about a periphery, a portion, or an entirety of drape 632. Additionally, or alternatively, drape 632 may be coupled to tissue site 620 via a double-sided drape tape, paste, hydrocolloid, hydrogel, and/or other sealing device or element, as illustrative, non-limiting examples.

Drape 632 may include an impermeable or semi-permeable, elastomeric material, as an illustrative, non-limiting example. In some implementations, drape 632 may be liquid/gas (e.g., moisture/vapor) impermeable or semi-permeable. Examples of elastomers may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane (PU), EVA film, co-polyester, and silicones. In some implementations, drape 632 may include the “V.A.C.® Drape” commercially available from KCI. Additional, specific non-limiting examples of materials of drape 632 may include a silicone drape, 3M Tegaderm® drape, and a polyurethane (PU) drape such as one available from Avery Dennison Corporation of Pasadena, Calif. An additional, specific non-limiting example of a material of the drape 632 may include a 30 micrometers (μm) matt polyurethane film such as the Inspire™ 2317 manufactured by Exopack™ Advanced Coatings of Matthews, N.C.

Referring to FIG. 6B, drape 632 includes or comprises a compound film 652 coupled to tissue site 620 by LSA 196. The compound film 652 of drape 632 includes a light blocking layer 692 and a drape layer 694. A layer or coating of LSA 196 is bonded to drape layer 694. Light blocking layer 692 may include or correspond to first film 142, light blocking layer 192, first film 242, first polymer layer 312, or light blocking layer 492. Drape layer 694 may include or correspond to second film 144, non-light blocking layer 194, second film 244, second polymer layer 314, or non-light blocking layer 494. In some implementations, drape 632 includes LSA 196 on only a portion of the compound film 652, such as a portion of the compound film 652 about a periphery of the drape 632.

Referring to FIG. 6A, manifold 634 is configured to be positioned on and/or near tissue site 620, and may be secured at the tissue site 620, such as secured by drape 632. The term “manifold” as used herein generally refers to a substance or structure that may be provided to assist in applying a pressure differential (e.g., positive-pressure differential) to, delivering fluids to, or removing fluids and/or exudate from a tissue site and/or target tissue. The manifold typically includes a plurality of flow channels or pathways that distribute fluids provided to and removed from the tissue site. In an illustrative implementation, the flow channels or pathways are interconnected to improve distribution of fluids provided to or removed from the tissue site. Manifold 634 may be a biocompatible material that may be capable of being placed in contact with the tissue site and distributing positive and/or negative-pressure to the tissue site. Manifold 634 may include, without limitation, devices that have structural elements arranged to form flow channels, such as foam, cellular foam, open-cell foam, porous tissue collections, liquids, gels, and/or a foam that includes, or cures to include, flow channels, as illustrative, non-limiting examples. Additionally, or alternatively, manifold may include polyethylene, a polyolefin, a polyether, polyurethane, a co-polyester, a copolymer thereof, a combination thereof, or a blend thereof.

In some implementations, manifold 634 is porous and may be made from foam, gauze, felted mat, or other material suited to a particular biological application. In a particular implementation, manifold 634 may be a porous foam and may include a plurality of interconnected cells or pores that act as flow channels. The foam (e.g., foam material) may be either hydrophobic or hydrophilic. As an illustrative, non-limiting example, the porous foam may be a polyurethane, open-cell, reticulated foam such as GranuFoam® material manufactured by Kinetic Concepts, Incorporated of San Antonio, Tex.

In some implementations, manifold 634 is also used to distribute fluids such as medications, antibacterials, growth factors, and other solutions to the tissue site. Other layers may be included in or on manifold 634, such as absorptive materials, wicking materials, hydrophobic materials, and hydrophilic materials. In an implementation in which the manifold 634 includes a hydrophilic material, manifold 634 may be configured to wick fluid away from tissue site 620 and to distribute positive-pressure to tissue site 620. The wicking properties of manifold 634 may draw fluid away from the tissue site 620 by capillary flow or other wicking mechanisms. An illustrative, non-limiting example of a hydrophilic foam is a polyvinyl alcohol, open-cell foam such as V.A.C. WhiteFoam® dressing available from Kinetic Concepts, Inc. of San Antonio, Tex. Other hydrophilic foams may include those made from polyether and/or foams that have been treated or coated to provide hydrophilicity.

In some implementations, manifold 634 is constructed from bioresorbable materials that do not have to be removed from tissue site 620 following use of the system 600. Suitable bioresorbable materials may include, without limitation, a polymeric blend of polylactic acid (PLA) and polyglycolic acid (PGA). The polymeric blend may also include without limitation polycarbonates, polyfumarates, and capralactones. Manifold 634 may further serve as a scaffold for new cell-growth, or a scaffold material may be used in conjunction with manifold 634 to promote cell-growth. A scaffold may be a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials. Although a manifold 634 is illustrated in FIG. 6A, in other implementations, dressing 616 does not include manifold 634. In such implementations, drape 632 of dressing 616 is coupled to connector 630.

Connector 630 includes a body 642 (e.g., housing) and a base 644, and is configured to be coupled to tube 614 via an interface 646 (e.g., a port). Base 644 is configured to be coupled to dressing 616. For example, base 644 may be coupled, such as via an adhesive, to drape 632 and/or manifold 634. In some implementations, base 644 comprises a flange that is coupled to an end of body 642 and/or is integrally formed with body 642. Connector 630, such as body 642, base 644, interface 646, or a combination thereof, may be made of rigid material and/or a semi-rigid material. In a non-limiting example, connector 630 may be made from a plasticized polyvinyl chloride (PVC), polyurethane, cyclic olefin copolymer elastomer, thermoplastic elastomer, poly acrylic, silicone polymer, or polyether block amide copolymer. In some implementations, connector 630 is formed of a semi-rigid material that is configured to expand when under a force, such as positive-pressure greater than or equal to a particular amount of pressure. Additionally or alternatively, connector 630 may be formed of a semi-rigid material that is configured to collapse when under a force, such as reduced-pressure less than or equal to a threshold pressure.

Body 642 includes one or more channels or one or more conduits that extend from and/or are coupled to interface 646. To illustrate, body 642 may include a primary channel configured to be coupled in fluid communication with a primary lumen (e.g., 621) of tube 614. The primary channel may be coupled to a cavity (e.g., a tissue cavity partially defined by body 642) having an aperture open towards manifold 634 (and/or towards tissue site 620). For example, the primary channel may include a first opening associated with interface 646 and a second opening (distinct from the aperture of the cavity) associated with the cavity. Thus, the primary channel may define a through channel of body 642 to enable fluid communication between interface 646 and tissue site 620.

Body 642 includes a channel (e.g., a through channel) having a first aperture open opposite dressing 616 and a second aperture open towards dressing 616. For example, the first aperture is located on an outer surface side (e.g., an ambient environment surface) of connector 630 and the second aperture is located on an inner surface side (e.g., a tissue facing side) of connector 630. The second aperture is configured to be coupled to one or more lumens of tube 614, such as coupled via the cavity. Illustrative, non-limiting examples of commercially available connectors include a “V.A.C. T.R.A.C.® Pad,” or “Sensa T.R.A.C.® Pad” available from Kinetic Concepts, Inc. (KCI) of San Antonio, Tex.

In some implementations, dressing 616 further includes a bandage and/or a wound closure device 660. For example, a bandage may be placed over a wound to protect the wound and a wound closure device 660 may be placed proximate to a wound to provide a force to maintain tissue in fixed position to promote wound closure. Each of the bandage and/or a wound closure device 660 may include compound film 652.

Light source 618 is configured to provide light to activate LSA 196 (photo initiators thereof) and cause LSA 196 to switch states. Light source 618 may include or correspond to the Sun, ambient lighting, a dedicated light device, such as an ultraviolet (UV) device, or a combination thereof.

An exemplary UV device is configured to generate/emit UV light to activate LSA 196 (photo initiators thereof) and cause LSA 196 to switch states. For example, UV device includes or corresponds to a UV light source configured to generate light or electromagnetic radiation having a wavelength of 10-500 nanometers, such as UV light to blue light. In some implementations, UV device may include or correspond to a UV torch. For example, UV torch may include one or more LEDs configured to generate incoherent light in the UV spectrum. In a particular implementation, UV torch generates light in a particular subspectrum of the UV spectrum, such as UVA or UVC.

In other implementations, UV device may include or correspond to a UV Laser, such as a gas laser, a laser diode, a solid-state laser, an excimer laser, or a combination thereof. In some implementations, UV laser is configured to generate coherent light (e.g., a laser beam) having electromagnetic radiation of UV wavelengths. For example, UV laser is a UVA laser (315-400 nm), a UVB laser (280-315 nm), a UVC laser (100-280 nm), or an extreme UV laser (10-121 nm).

During operation of system 600, dressing 616 is coupled to tissue site 620 over a wound. Additionally, dressing 616 is coupled to device 610 via tube 614. In some implementations, prior to coupling the dressing 616 to the tissue site 620, a bandage or a wound closure device 660 is coupled to tissue site 620 proximate to a wound. The dressing 616 is then coupled over the bandage or wound closure device 660. One or more of the dressing 616 or over the bandage or wound closure device 660 is coupled to tissue 620 site via compound film 652. To illustrate, light switchable adhesive 196 of the compound film 652 bonds the dressing 616, the bandage or wound closure device 660, or both to the tissue site 620 responsive to pressure. In a particular implementation when the compound film 652 is included in or corresponds to drape 632, and the compound film 652 may seal a portion of tissue site 620, such as an interior volume of dressing 616.

A pressure differential, such as positive-pressure, can be generated and/or applied to dressing 616 (e.g., the interior volume of dressing 616) by a pressure source associated with device 610. When positive-pressure is generated and/or applied to dressing 616, fluid or medication from device 610, such as from canister 612, may be transported to dressing 616. Furthermore, in some implementations, reduced-pressure can be applied to dressing 616 (e.g., the interior volume of dressing 616 or a second interior volume of the dressing 616) by a reduced-pressure source associated with device 610. When reduced-pressure is applied to dressing 616 (e.g., when vacuum pressure is generated, fluid, exudate, or other material within dressing 616 may be transported to canister 612 of device 610.

After operation, such as completion of therapy, system 600 may be disconnected and components thereof removed from tissue site 620. For example, light blocking layer 692 of compound film 652 may be removed from drape layer 694 exposing LSA 196 thereof to light. The LSA 196 disposed on drape layer 694 may transition from a first high tack state to a second low tack state by cross-linking. Accordingly, drape 632, and thus dressing 616, can be easily removed from tissue site 620. In some implementations where a bandage/wound closure device 660 is used and where the bandage/wound closure device 660 includes a compound film 652, the LSA 196 can be activated by light such as UV light. To illustrate, the light blocking layer 696 of the compound film 652 of the bandage/wound closure device 660 may be removed from drape layer 694 exposing LSA 196 to light. Similarly, the bandage/wound closure device 660 can be easily removed from tissue site 620.

Thus, dressing 616, bandage/wound closure device 660, or both, can be adhered to a patient with a compound film and can be transitioned to a low tack state to be painlessly and easily removed. In some implementations, the compound film includes a light-blocking layer that includes polyurethane and does not include an adhesive or relatively low breathable layer between the light blocking layer and the drape layer. Accordingly, the compound film enables higher breathability and wearability, as compared to compound films with dissimilar materials.

Referring to FIG. 7, a block diagram of a manufacturing system, system 700, for making components including compound films including (e.g., coating in) LSA. In the example illustrated in FIG. 7, system 700 includes a control system 710, a film processing system 712, a film lamination system 714, and a LSA coating system 716. Control system 710 is configured to control one or more of systems 712-716, as described further herein.

Film processing system 712 includes one or more extruders 720, one or more dies 722, and optionally includes one or more heaters (e.g., heating devices). Film processing system 712 may include or correspond to extruder 214 and die 216 of FIG. 2. Film processing system 712 is configured to generate one or more films 742, 744 from one or more polymers 726. For example, film processing system 712 may be configured to generate film 142, film 144, or both. As another example, film processing system 712 may be configured to generate film 242, film 244, or both. Film processing system 712 may include or correspond to an extrusion film system. For example, film processing system 712 receives or generates pellets or resin of one or more polymers 726 or receives a polymer composition (e.g., polymer composite) including one or more polymers 726, and film processing system 712 produces extrudate of a polymer material based on the received polymer material. The extrudate may have the form of or may be formed into a film of polymer material (i.e., a polymer film of a polymer composition). As an illustrative example, film processing system 712 may include or correspond to a melt-compounding system or a melt-blend combiner.

Film lamination system 714 includes one or more rollers 730, and optionally one or more heaters 732 and/or cooling equipment 734. The one or more heaters may include or correspond to steam heaters or electric heating devices, as illustrative, non-limiting examples. Film lamination system 714 is configured to join or laminate two or more films (e.g., 742, 744) to generate a compound film 752. For example, film lamination system 714 is configured to join film 142 and film 144 to generate compound film 152. As another example, film lamination system 714 is configured to join film 242 and film 244 to generate compound film 252. In some implementations, film lamination system 714 includes or corresponds to a film-to-film lamination system, such as system 100. For example, film lamination system 714 includes a plurality of rollers 730 configured to receive rolls of multiple films, such as 142, 144, and is configured to feed and press the films together to form the compound film 752. In a particular implementation, the film lamination system 714 presses together another film and/or post processes the compound film 752. For example, a third film (e.g., support layer 490) may be pressed together with the other two films 742, 744 before, during, or after the other two films 742, 744 are pressed together. As another example, the compound film 752 may be perforated, cut, partially repressed, heated, cooled, etc., based on a desired peel strength and/or based on application of LSA, such as LSA 196 (e.g., 754).

In other implementations, film lamination system 714 is configured to generate one or more of the films used to generate the compound film 752. For example, film lamination system 714 may include or correspond to an extrusion-cast film system, such as system 200, and may include a melt-compounding system or a melt-blend combiner. To illustrate, film lamination system 714 is configured to generate the first film 742 or the second film 744 using extruder 736. Film lamination system 714 may generate the first film 742 or the second film 744 similar to the film processing system 712. The first film 742 or the second film 744 may include or correspond to one of films or layers 142, 144, 192, 194, 312, 314, 492, 494, 692, or 694.

LSA coating system 716 is configured to apply LSA 754 to or form a coating of LSA on the compound film 752. For example, LSA coating system 716 is configured to apply or selectively apply LSA 754 to compound film 752, as described with reference to FIGS. 5A-5J. LSA coating system 716 includes an applicator 760 and LSA 754. Applicator 760 may be configured to apply the LSA 754 to second film 742 of compound film 752 in a pattern, i.e., apply a pattern of LSA 754. For example, applicator 760 selectively applies the LSA 754 according to patterns 502-518, or applicator 760 applied a coating of LSA 754 and a removal device (e.g., a blade, a scraper, a wiper, a roller, etc.) selectively removes a portion of the coating. In some implementations, the applicator 760 is a die (e.g., a slot die), a roller, a patterned roller, a spray nozzle, etc.

LSA coating system 716 may optionally include one or more heaters 762, curing devices 764, mixing devices 766, or a combination thereof. The one or more heaters 762 and mixing devices 766 may be configured to heat and mix LSA 754 prior to application and/or delivery to applicator 760. The one or more curing device 764 may be configured to apply heat or light to the LSA 754 after application by the applicator 760. The compound film 752 may include or correspond to compound film 152, compound film 252, compound film 352A, compound film 352B, compound film 402, compound film 552, or compound film 652.

Although listed as separate systems, one or more of systems 712-716 may be incorporated into a single system. For example, film processing system 712 and film lamination system may be incorporated into a single system, as described with reference to FIG. 2. As another example, film lamination system 714 and LSA coating system 716 may be incorporated into a single system. Additionally, system 700 may include one or more other systems, such as a cover film lamination system, a support layer lamination system, a post-processing system, a packing system, a sterilization system, or a combination thereof. The post-processing system may be configured to cut and/or form the compound film 752 into shapes and add features to the compound film 752. For example, the post-processing system may modify the compound film to add tabs (e.g., 344) or sidewalls (e.g., 444).

Control system 710 includes one or more interfaces 770, one or more controllers, such as a representative controller 772, and one or more input/output (I/O) devices 778. Interfaces 770 may include a network interface and/or a device interface configured to be communicatively coupled to one or more other devices, such as film processing system 712, film lamination system 714, or LSA coating system 716. For example, interfaces 770 may include a transmitter, a receiver, or a combination thereof (e.g., a transceiver), and may enable wired communication, wireless communication, or a combination thereof. Although control system 710 is described as a single electronic device, in other implementations system 700 includes multiple electronic devices. In such implementations, such as a distributed control system, the multiple electronic devices each control a sub-system of system 700, such as film processing system 712, film lamination system 714, or forming system 716.

The one or more controllers (e.g., controller 772) includes one or more processors and one or more memories, such as representative processor 774 and memory 776. The one or more controllers may include or correspond to a film processing controller, a film lamination controller, an LSA application controller, or a combination thereof. For example, film processing controller (e.g., processor 774) may be configured to generate and/or communicate one or more control signals 782 to film processing system 712. Film lamination controller may be configured to control (or regulate) an environment, such as an air quality, temperature, and/or pressure, within film lamination system 714 (e.g., a laminating chamber or zone or an extruder thereof) and/or delivery/injection of materials into film lamination system 714. For example, film lamination controller may be configured to generate and/or communicate one or more control signals 782, such as environment control signals, ingredient delivery control signals, or a combination thereof, to film lamination system 714.

LSA application controller may be configured to control (or regulate) an environment, such as a temperature (e.g., heat) and/or pressure of LSA 754, applicator 760, or both, within LSA coating system 716 (e.g., applicator 760 thereof) and/or delivery/injection of LSA 754 into LSA coating system 716 (e.g., applicator 760 thereof). For example, application controller may be configured to generate and/or communicate one or more control signals 782, such as environment control signals, ingredient delivery control signals, or a combination thereof, to LSA coating system 716.

Memory 776, such as a non-transitory computer-readable storage medium, may include volatile memory devices (e.g., random access memory (RAM) devices), nonvolatile memory devices (e.g., read-only memory (ROM) devices, programmable read-only memory, and flash memory), or both. Memory 776 may be configured to store instructions 792, one or more thresholds 796, and one or more data sets 798. Instructions 792 (e.g., control logic) may be configured to, when executed by the one or more processors 774, cause the processor(s) 774 to perform operations as described further here. For example, the one or more processors 774 may perform operations as described with reference to FIGS. 1A, 2, 5A-5J, 8, and 9. The one or more thresholds 796 and one or more data sets 798 may be configured to cause the processor(s) 774 to generate control signals. For example, the processors 774 may generate and send control signals responsive to receiving sensor data from one or more of system 712-716, such as exemplary sensor data 784 from LSA coating system 716. The temperature or ingredient flow rate can be adjusted based on comparing sensor data to one or more thresholds 796, one or more data sets 798, or a combination thereof.

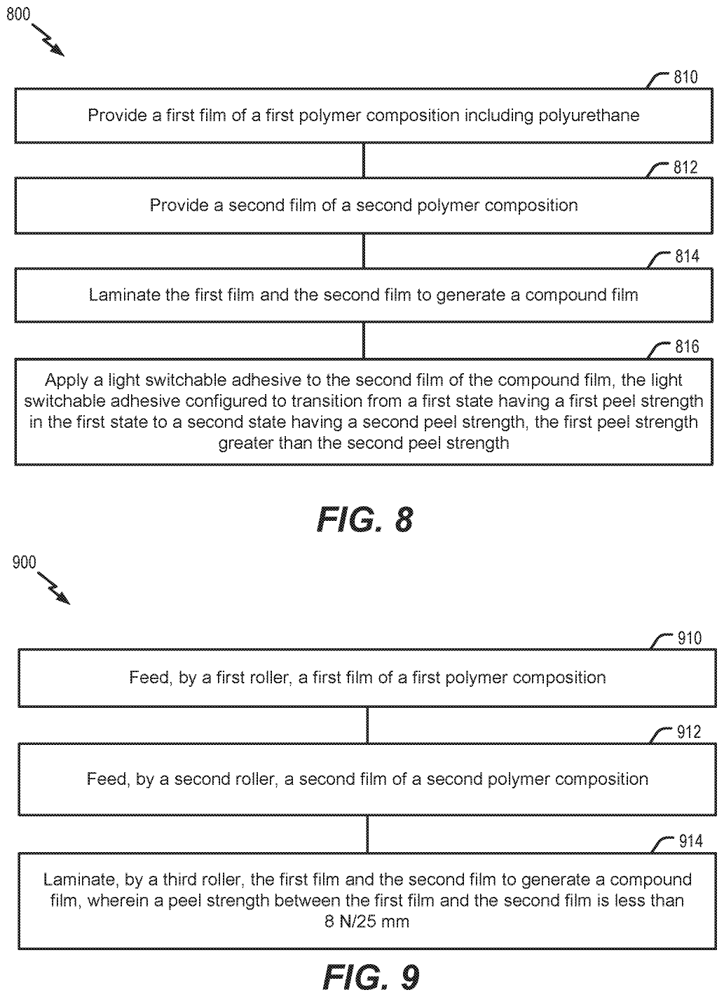

In some implementations, processor 774 may include or correspond to a microcontroller/microprocessor, a central processing unit (CPU), a field-programmable gate array (FPGA) device, an application-specific integrated circuits (ASIC), another hardware device, a firmware device, or any combination thereof. Processor 774 may be configured to execute instructions 792 to initiate or perform one or more operations described with reference to FIG. 1A, FIG. 2, and/or one more operations of the methods of FIGS. 8 and 9.

The one or more I/O devices 778 may include a mouse, a keyboard, a display device, the camera, other I/O devices, or a combination thereof. In some implementations, the processor(s) 774 generate and send control signals responsive to receiving one or more user inputs via the one or more I/O devices 778.

Control system 710 may include or correspond to an electronic device such as a communications device, a mobile phone, a cellular phone, a satellite phone, a computer, a tablet, a portable computer, a display device, a media player, or a desktop computer. Additionally, or alternatively, the control system 710 may include a personal digital assistant (PDA), a monitor, a computer monitor, a television, any other device that includes a processor or that stores or retrieves data or computer instructions, or a combination thereof.

During operation of system 700, film processing system 712 forms one or more of first film 742 or second film 744. For example, film processing system 712 generates one or more of first film 742 or second film 744 using polymer(s) 726. To illustrate, controller 772 may send one or more control signals 782 to film processing system 712. The control signals 782 may include signals configured to cause film processing system 712 to mix or blend polymer(s) 726 (e.g., resin or pellets thereof), and optionally additive(s), to form a polymer composition or blend in extruder 720. To illustrate, control system 710 may send one or more signals 782 (e.g., environment control signals) to film processing system 712 to adjust conditions (e.g., heat, pressure, air quality) of the film processing system 712 or conditions (e.g., viscosity, temperature, etc.) of the polymer composition. Additionally or alternatively, control system 710 may send one or more control signals 782 (e.g., ingredient delivery control signals) to film processing system 712 to adjust rates and or amounts of polymer(s) 726, one or more additives, or a combination thereof.

In some implementations, heater 724 provides heat to extruder 720 or to polymer(s) 726 prior to delivery to the extruder 720. The polymer composition or blend is extruded by extruder 720 via a die 722 to form extrudate. The extrudate may include or correspond to a film of polymer material, i.e., a film of a polymer composition.

Additionally, the control signals 782 may include signals configured to cause film processing system 712 to cool the extrudate to form one of the films. In a particular implementation, film processing system 712 includes multiple extruders 720 and dies 722 to produce multiple films, such as 742, 744. The polymer compositions of each film may have the same main ingredient, i.e., a same or similar material makes up a majority (e.g., largest ingredient by weight or 50.1 percent by weight). To illustrate, each of film 742, 744 may have polyurethane (PU) as its majority ingredient or majority polymer. Thus, the first polymer composition may be substantially similar to the second polymer composition. To illustrate, each of the first polymer composition and the second polymer composition may include similar polymer materials and/or additives. In a particular implementation, the second polymer composition includes two or more materials of the first polymer composition and includes a first concentration (e.g., by weight) of the two or more materials within plus or minus 20 percent of a second concentration of the two or more materials of the first polymer composition.

After generation of the first film 742 and/or the second film 744, the film(s) 742, 744 are provided to film lamination system 714. Film lamination system 714 generates compound film 752 based on removably coupling films 742, 744 together. For example, film lamination system 714 bonds or laminates films 742, 744 via application of pressure and optionally heat to form compound film 752. In some implementations, the film lamination system 714 includes one or more rollers 730 to laminate the films, as described with reference to FIG. 1A.

To illustrate, controller 772 may send one or more control signals 782 to film lamination system 714 configured to cause film lamination system 714 to laminate films 742, 744 to form a compound film 752 using rollers 730. To illustrate, control system 710 may send one or more signals 782 (e.g., environment control signals) to film lamination system 714 to adjust conditions (e.g., heat, pressure, air quality) of the film lamination system 714 (e.g., rollers 730) or conditions of the polymer composite (viscosity, temperature, etc.). Additionally or alternatively, control system 710 may send one or more control signals 782 (e.g., feed speed control signals) to film processing system 712 to adjust rates and or amounts of films 742, 744, rpms of roller 730, or a combination thereof.

In a particular implementation, the film lamination system 714 includes an extrusion cast film system which forms one of the films 742 or 744 during the laminating process. In such implementations, film lamination system 714 may form a film similar to as described with reference to the film processing system 712.

After formation of compound film 752, the compound film 752 is provided to LSA coating system 716 and LSA coating system 716 applies or forms a coating of LSA 754 on the compound film 752. For example, LSA coating system 716 may form a coating or film of LSA 754 on the compound film 752 via selective application. To illustrate, control system 710 may send or more control signals to control delivery (e.g., application) of LSA 754 to applicator 760 of LSA coating system 716, LSA 754 to compound film 752 via applicator 760, or both. In other implementations, LSA coating system 716 forms the LSA 754 on the compound film 752 via selective removal. To illustrate, control system 710 may send or more control signals to control removal (e.g., scraping or removing) of LSA 754 from compound film 752.

In some implementations, coating system 716 may receive control signals 782 to control a heater 762 and/or a mixing device 766 to heat and/or mix the LSA 754 prior to delivery of LSA 754 to applicator 760. Additionally, LSA coating system 716 may receive control signals 782 to control a curing device 764 to cure the LSA 754 applied to the compound film 752.

Thus, system 700 of FIG. 7 produces compound films with reduced intralayer peel strengths and increased breathability. Accordingly, the present disclosure overcomes the existing challenges of forming (e.g., commercial manufacture of) high breathability compound films, such as PU/PU films, that are capable of use with conventional LSA.

FIG. 8 illustrates a method 800 of manufacturing a compound film. The method 800 may be performed at or by system 100 (e.g., the rollers thereof), system 200 (e.g., the rollers thereof), or the system 700 (e.g., systems 712 and/or 714 thereof).

Method 800 includes providing a first film of a first polymer composition including polyurethane, at 810. For example, the first film may include or correspond to first film 142, first film 242, or first film 742, and the first polymer composition may include or correspond to first polymer layer 312. To illustrate, a roll of first film 142 is received at roller 122. As another illustration, first film 242 is extruded by extruder 214 via die 216.

Method 800 also includes providing a second film of a second polymer composition, at 812. For example, the second film may include or correspond to second film 144, second film 244, or second film 744, and the second polymer composition may include or correspond to second polymer layer 314. To illustrate, a roll of second film 144 is received at roller 132. In some implementations, the first and second polymer compositions include polyurethane (PU). As another illustration, second film 244 is extruded by an extruder (e.g., 214) via a die (e.g., 216). In some implementations, the first polymer composition may be substantially similar to the second polymer composition, as described with reference to FIG. 7. Additionally, or alternatively, the first polymer composition and the second polymer composition each comprise a same or similar majority material. A majority material may include a largest polymer ingredient by weight or a polymer that is at least 50.1 percent by weight.

Method 800 includes laminating the first film and the second film to generate a compound film, at 814. For example, the compound film may include or correspond to compound film 152, compound film 252, compound film 352A, compound film 352B, compound film 402, compound film 552, compound film 652, or compound film 752. To illustrate, a roller, a press, a commercial document laminator, or another device that applies pressure, and optionally heat, removably bonds films 142, 144 together to form compound film 152. In some implementations, a peel strength between the first film and the second film is less than 8 N/25 mm. Additionally, or alternatively, a peel strength between the light switchable adhesive and a tissue is greater than or equal to 8 N/25 mm.

Method 800 further includes applying a light switchable adhesive to the second film of the compound, the light switchable adhesive configured to transition from a first state having a first peel strength in the first state to a second state having a second peel strength, the first peel strength greater than the second peel strength, at 816. A third peel strength between the first layer and the second layer is less than the second peel strength between the light switchable adhesive in the first state and a bond site. For example, the light switchable adhesive may include or correspond to LSA 196, LSA 396, or LSA 754. To illustrate, applicator 760 applies LSA 754 to second film 744, as described with reference to FIG. 7.

In some implementations, method 800 further comprises coupling a cover film to the light switchable adhesive and/or coupling a support layer including a third polymer composition to the first film. In a particular implementation, the third polymer composition is different from the first polymer composition, and the support layer is coupled to the first film prior to the first film being laminated to the second film. For example, the first film (e.g., 142) and the third film (e.g., film corresponding to support layer 490) may be coextruded films and the co-extruded compound film may be bonded with the second film (e.g., 144).

Thus, method 800 describes method of forming a compound film that includes light switchable adhesive and that is suitable for use with light switchable adhesive. The compound film is configured to have a lower peel strength as compared to compound films of similar materials manufactured by co-extrusion processes and to compound films bonded together with adhesives. Accordingly, such compound films described herein can be used with LSA. Additionally, the compound film enables multiple layers of the compound film, such as a light blocking layer and a non-light blocking layer (e.g., LSA host layer), to have and increased breathability and wearability as compared to layers of conventional films that are used in LSA applications, i.e., films with a sufficiently low peel strength to have a peel strength less than a peel strength of a bond created by the LSA. As an illustrative example, the compound film enables a higher water vapor transfer rate and/or a higher oxygen transfer rate (e.g., permeability) as compared to compound films that include one high breathability layer and one relatively lower breathability layer, such as PE/PU films. Therefore, the compound film is suitable for use in medical devices, such as bandages, drapes, dressings, and wound closures. The compound film enables medical devices to have reduced layers and increased breathability as compared to conventional compound film, thereby avoiding or limiting maceration and tissue damage at tissue site and patient discomfort. Accordingly, the compound film may enable improved wound care and therapy and increased wear times of medical devices, thereby advancing patient comfort and confidence in the treatment.

FIG. 9 illustrates a method 900 of manufacturing a compound film. The method 900 may be performed at or by system 100 (e.g., the rollers thereof), system 200 (e.g., the rollers thereof), or the system 700 (e.g., systems 712 and/or 714 thereof).

Method 900 includes feeding, by a first roller, a first film of a first polymer composition, at 910. For example, the first roller may include or correspond to roller 122, roller 132, roller 222, roller 224, roller 232, another feed roller, or a roller of a rotary press. The first film may include or correspond to first film 142, first film 242, or first film 742, and the first polymer composition may include or correspond to first polymer layer 312. To illustrate, roller 122 feeds first film 142 to roller 124.

Method 900 also includes feeding, by a second roller, a second film of a second polymer composition, at 912. For example, the second roller may include or correspond to roller 122, roller 132, roller 222, roller 224, roller 232, another feed roller, or a roller of a rotary press. The second film may include or correspond to second film 144, second film 244, or second film 744, and the second polymer composition may include or correspond to second polymer layer 314. To illustrate, roller 132 feeds second film 144 to roller 134. In some implementations, the first polymer composition may be substantially similar to the second polymer composition, as described with reference to FIG. 7. In a particular implementation, the first and second polymer composition include polyurethane (PU). Additionally, or alternatively, the first polymer composition and the second polymer composition each comprise a same or similar majority material. A majority material may include a largest polymer ingredient by weight or a polymer that is at least 50.1 percent by weight.

Method 900 further includes laminating, by a third roller, the first film and the second film to generate a compound film, at 914. A peel strength between the first and the second film is less than 8 N/25 mm. For example, the third roller may include or correspond to roller 124, roller 134, patterned roller 172, roller 226, roller 234, another compression roller, a heated roller, another pattern roller, or a roller of a rotary press. The compound film may include or correspond to compound film 152, compound film 252, compound film 352A, compound film 352B, compound film 402, compound film 552, compound film 652, or compound film 752. To illustrate, rollers 124, 134 apply pressure to films 142, 144 to generate compound film 152, as illustrated in FIG. 1A.

Thus, method 900 describes a method of forming a compound film with a reduced peel strength and/or a compound film that is suitable for use with light switchable adhesive. The compound film is configured to have a lower peel strength as compared to compound films of similar materials manufactured by co-extrusion processes and to compound films bonded together with adhesives. Accordingly, such compound films can be used with LSA. Additionally, the compound film enables multiple layers of the compound film, such as a light blocking layer and a non-light blocking layer (e.g., LSA host layer), to have and increased breathability and wearability as compared to layers of conventional films that are used in LSA applications, i.e., films with a sufficiently low peel strength to have a peel strength less than a peel strength of a bond created by the LSA. As an illustrative example, the compound film enables a higher water vapor transfer rate and/or a higher oxygen transfer rate (e.g., permeability) as compared to compound films that include one high breathability layer and one relatively lower breathability layer, such as PE/PU films. Therefore, the compound film is suitable for use in medical devices, such as bandages, drapes, dressings, and wound closures. The compound film enables medical devices to have reduced layers and increased breathability as compared to conventional compound film, thereby avoiding or limiting maceration and tissue damage at tissue site and patient discomfort. Accordingly, the compound film may enable improved wound care and therapy and increased wear times of medical devices, thereby advancing patient comfort and confidence in the treatment.

It is noted that one or more operations described with reference to one of the methods of FIGS. 8-9 may be combined with one or more operations of another of FIGS. 8-9. For example, one or more operations of method 800 may be combined with one or more operations of method 900. Additionally, or alternatively, one or more operations described above with reference to FIGS. 1A, 1B, 2, 3A-3D, 4A-4F, 5A-5J, 6A, 6B and 7 may be combined with one or more operations of FIG. 8, FIG. 9, or a combination of FIGS. 8 and 9.

The above specification and examples provide a complete description of the structure and use of illustrative examples. Although certain aspects have been described above with a certain degree of particularity, or with reference to one or more individual examples, those skilled in the art could make numerous alterations to aspects of the present disclosure without departing from the scope of the present disclosure. As such, the various illustrative examples of the methods and systems are not intended to be limited to the particular forms disclosed. Rather, they include all modifications and alternatives falling within the scope of the claims, and implementations other than the ones shown may include some or all of the features of the depicted examples. For example, elements may be omitted or combined as a unitary structure, connections may be substituted, or both. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one example or may relate to several examples. Accordingly, no single implementation described herein should be construed as limiting and implementations of the disclosure may be suitably combined without departing from the teachings of the disclosure.

The previous description of the disclosed implementations is provided to enable a person skilled in the art to make or use the disclosed implementations. Various modifications to these implementations will be readily apparent to those skilled in the art, and the principles defined herein may be applied to other implementations without departing from the scope of the disclosure. Thus, the present disclosure is not intended to be limited to the implementations shown herein but is to be accorded the widest scope possible consistent with the principles and novel features as defined by the following claims. The claims are not intended to include, and should not be interpreted to include, means-plus- or step-plus-function limitations, unless such a limitation is explicitly recited in a given claim using the phrase(s) “means for” or “step for,” respectively.

1. A compound film comprising:
a first layer of a first polymer composition including polyurethane;
a second layer of a second polymer composition, the second layer removably coupled to the first layer; and
a light switchable adhesive coupled to the second layer and configured to transition from a first state to a second state, the light switchable adhesive has a first peel strength in the first state that is greater than a second peel strength of the light switchable adhesive in the second state, wherein a third peel strength between the first layer and the second layer is less than the second peel strength between the light switchable adhesive in the first state and a bond site.

2. The compound film of claim 1, wherein:
the light switchable adhesive comprises a layer of light switchable adhesive in contact with the second layer; and
the second polymer composition includes polyurethane.

3. The compound film of any of claim 1, wherein the third peel strength between the first layer and the second layer is between 0.5 N/25 mm to 3 N/25 mm.

4. The compound film of claim 3, wherein the third strength between the first layer and the second layer is between 1.5 N/25 mm to 2.5 N/25 mm.

5. The compound film of claim 4, wherein:
the bond site is a tissue site; and
the light switchable adhesive is configured to generate the first peel strength of greater than 3 N/25 mm between the light switchable adhesive and the tissue site within 2 hours after application of the light switchable adhesive to the tissue site.

6-7. (canceled)

8. The compound film of claim 1, wherein:
the first layer is configured to block or filter UV light to blue light; and
the second layer is configured to pass UV light to blue light, or both.

9. (canceled)

10. The compound film of claim 1, wherein:
the second layer is configured to pass visible light; and
the first layer is configured to block or filter visible light.

11. The compound film of claim 1, further comprising:
a support layer including a third polymer material coupled to the first layer, the support layer having a first rigidity that is greater than a second rigidity of the first layer; and
a cover film removably coupled to the light switchable adhesive.

12. (canceled)

13. The compound film of claim 1, wherein the light switchable adhesive, the first layer, the second layer, or a combination thereof, define a plurality of perforations.

14. (canceled)

15. A method comprising:
providing a first film of a first polymer composition including polyurethane;
providing a second film of a second polymer composition;
laminating the first film and the second film to form a compound film; and
applying a light switchable adhesive to the second film of the compound film, the light switchable adhesive configured to transition from a first state having a first peel strength in the first state to a second state having a second peel strength, the first peel strength greater than the second peel strength, and a third peel strength between the first layer and the second layer is less than the second peel strength between the light switchable adhesive in the first state and a bond site.

16. The method of claim 15, wherein:
providing the first film includes feeding, by a first roller, the first film; and
providing the second film includes feeding, by a second roller, the second film.

17. The method of claim 16, wherein laminating the first film and the second film to form the compound film includes applying, by a third roller, heat, pressure, or both.

18. The method of claim 17, further comprising applying heat to the third roller by a heat element that is distinct from the third roller.

19. The method of claim 17, further comprising applying heat, by a heat element that is distinct from the third roller, to the first film, the second film, or both.

20. The method of claim 15, wherein applying the light switchable adhesive to the second film includes applying a coating of light switchable adhesive by a roller, a slot die, or a spray nozzle.

21. (canceled)

22. The method of claim 15, further comprising:
coupling a cover film to the light switchable adhesive; and
coupling a support layer including a third polymer composition to the first film.

23. The method of claim 22, wherein:
the third polymer composition is different from the first polymer composition; and
the support layer is coupled to the first film prior to the first film being laminated to the second film.

24. The method of claim 15, further comprising forming perforations in the light switchable adhesive, the first film, the second film, or a combination thereof.

25. (canceled)

26. The method of claim 15, wherein providing the first film includes extrusion casting the first polymer composition to form the first film.

27. A system for manufacturing a compound film, the system comprising:
one or more first rollers associated with a first film of a first polymer composition including polyurethane; and
one or more second rollers associated with a second film of a second polymer composition,
wherein the one or more first roller, the one or more second rollers, or a combination thereof are configured to laminate the first film and the second film to form a compound film, and
wherein a peel strength between the first film and the second film of the compound film is less than 8 N/25 mm.

28. The system of claim 27, further comprising an applicator configured to apply a light switchable adhesive to the second film.

29. The system of claim 27, wherein the one or more first rollers, the one or more second rollers, or both, include a roller selected from the group consisting of a stainless steel roller, a Teflon roller, and a silicone coated roller.

30. The system of claim 27, further comprising a controller configured to control at least one of the one of more first rollers, the one or more second rollers, or a combination thereof.

31. The system of claim 27, further comprising an extrusion cast film system including an extruder and a die and configured to generate the first film or the second film.

32. The system of claim 27, further comprising a coating system configured to apply a light switchable adhesive to the second film.

33. The system of claim 27, wherein the coating system comprises a roller, a platten, or a die configured to apply the light switchable adhesive.

34. The system of claim 27, wherein:
at least one of the one of more first rollers, the one or more second rollers, or both, comprise a patterned roller; and
the coating system is configured to the apply the light switchable adhesive to the second film of the compound film in a pattern.

35. The system of claim 27, further comprising:
a steam heater or an electric heating device configured to heat at least one of the one of more first rollers, the one or more second rollers, or a combination thereof; and
a perforation device configured to generate perforations in the first film, the second film, or both.

36-42. (canceled)
